(19) United States
(12) Patent Application Publication
(10) Pub. No.: US 2005/0215772 A1
(43) Pub. Date: Sep. 29, 2005

(54) Furanone derivatives and methods of making same

(76) Inventor: Naresh Kumar, New South Wales (AU)

Correspondence Address: NIXON & VANDERHYE, PC, 901 NORTH GLEBE ROAD, 11TH FLOOR, ARLINGTON, VA, 22203, US

(21) Appl. No.: 10/525,231

(22) PCT Filed: Aug. 19, 2003
(86) PCT No.: PCT/AU03/01053

§ 371 Date: Apr. 22, 2005

(30) Foreign Application Priority Data
Aug. 19, 2002 (AU) 2002950862

(51) Int. Cl. C07H 19/22; C07H 19/048; C07D 207/24

(52) U.S. Cl. 530/409; 536/27.1; 536/28.1; 548/543

(57) ABSTRACT

Novel synthesis methods, to the products of such novel methods, and to uses of these products. In particular, the present invention provides methods for the reactions of furanones, in particular fimbrolides, with amines. The invention has particular application in the synthesis of halogenated 1,5-dihydro-pyrrol-2-one, 5-halomethylene substituted 1,5-dihydropyrrol-2-ones (lactam analogues of fimbriolides), 5-amino substituted furanones and 5-aminomethylene-2(5H)-furanones and their synthetic analogues. The invention also relates to novel compounds and uses thereof.

### TECHNICAL FIELD

The present invention relates to novel synthesis methods, to the products of such novel methods, and to uses of these products. In particular, the present invention provides methods for the reactions of furanones, in particular fimbrolides, with amines. The invention has particular application in the synthesis of halogenated 1,5-dihydro-pyrrol-2-one, 5-halomethylene substituted 1,5-dihydropyrrol-2-ones (lactam analogues of fimbrolides), 5-amino substituted furanones and 5-aminomethylene-2(5H)-furanones and their synthetic analogues. The invention also relates to novel compounds and uses thereof.

### BACKGROUND

Fimbrolides (halogenated 5-methylene-2(5H)-furanones) possess a wide range of important biological properties including antifungal and antimicrobial properties (see WO 96/29392 and WO 99/53915, the disclosures of which are incorporated herein by cross-reference). These metabolites can be isolated from red marine algae Delisea fimbriata, Delisea elegans and Delisea pulchra.

Despite their biological activity very few hetero atom containing analogues of these molecules have been reported in the literature. The majority of the published syntheses of fimbrolides focus on the preparation of naturally occurring fimbrolides themselves. Recently we have developed methods that yield both the natural and non-natural fimbrolides in good yields (see WO 99/54323 and WO 0200639 the disclosure of which is incorporated herein by cross-reference).

We have now found that, surprisingly, fimbrolides undergo reactions with amines under mild conditions. We have found this discovery to be particularly useful in the synthesis of 5-hydroxy-5-alkyl substituted 1,5-dihydro-pyrrol-2-one, 5-amino-5-alkyl substituted 2(5H)-furanones and 5-aminomethylene substituted 2(5H)-furanones. Furthermore 5-hydroxy-5-halomethyl substituted 1,5-dihydro-pyrrol-2-one generated under these conditions can be dehydrated to yield 5-halomethylene substituted 1,5-dihydropyrrol-2-ones (lactam analogues of fimbrolides), and the 5-amino-5-bromomethyl substituted 2(5H)-furanones can be dehydrobrominated to yield a range of 5-aminomethylene substituted 2(5H)-furanones. These furanones can be further functionalised to yield a range of novel analogues.

### SUMMARY OF THE INVENTION

In a first aspect, the present invention provides a method for the preparation of compound of formula II 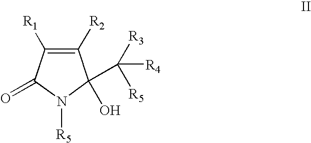
wherein R₁ and R₂ are independently selected from the group H, halogen, substituted or unsubstituted alkyl, substituted or unsubstituted alkoxy, substituted or unsubstituted oxoalkyl, substituted or unsubstituted alkenyl, aryl or substituted or unsubstituted arylalkyl, optionally interrupted by one or more hetero atoms, straight chain or branched chain, hydrophilic or fluorophilic;
R₃ and R₄ are independently selected from the group H, halogen, substituted or unsubstituted alkyl, substituted or unsubstituted alkoxy, substituted or unsubstituted aryl or substituted or unsubstituted arylalkyl;
R₅ is selected from the group consisting of H, hydroxy, substituted or unsubstituted alkyl, substituted or unsubstituted alkoxy, substituted or unsubstituted oxoalkyl, substituted or unsubstituted alkenyl, substituted or unsubstituted aryl or substituted or unsubstituted arylalkyl, optionally interrupted by one or more hetero atoms, straight chain or branched chain, hydrophilic or fluorophilic, or
forms part of an amino acid, or
is a nucleoside, an oligomer, a polymer, a dendrimer, a substrate or a surface;
the method comprising reacting a compound of formula I 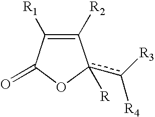
wherein R₁ and R₂ are independently H, halogen, alkyl, substituted or unsubstituted alkoxy, substituted or unsubstituted oxoalkyl, substituted or unsubstituted alkenyl, substituted or unsubstituted aryl or substituted or unsubstituted arylalkyl, optionally interrupted by one or more hetero atoms, straight chain or branched chain, hydrophilic or fluorophilic;
R₃ and R₄ are independently H, halogen, alkyl, substituted or unsubstituted alkoxy, substituted or unsubstituted aryl or arylalkyl; and R is hydroxy, halogen; and 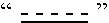
   represents a single bond, in which case R is absent, or a double bond, provided that at least one of R₁, R₂, R₃ and R⁴ is halogen,
with a compound of formula R₅NH₂
wherein R₅ is selected from the group consisting of H, substituted or unsubstituted alkyl, hydroxy, substituted or unsubstituted alkoxy, substituted or unsubstituted oxoalkyl, substituted or unsubstituted alkenyl, substituted or unsubstituted aryl or substituted or unsubstituted arylalkyl, optionally interrupted by one or more hetero atoms, straight chain or branched chain, hydrophilic or fluorophilic, or
forms part of an amino acid, or
is a nucleoside, an oligomer, a polymer, a dendrimer, a substrate or a surface.

The reaction may optionally be carried out in the presence of solvent.

Preferably, in the compound of formula II, at least one of R₁, R₂, R₃ and R₄ is halogen.

In the structural formulae described herein, a particular geometry is not to be taken as specified. For example, the formulae covers both Z- and E-isomers.

The reaction may be performed in the presence or absence of a solvent. The solvent may be any suitable solvent. Preferable solvents in the present invention include alkyl acetates, aromatic hydrocarbons, chlorinated alkanes, cyclic or open chain ethers such as tetrahydrofuran, diethyl ether, dioxane, and C₁-C₃ acids. More preferably, the solvents are aromatic hydrocarbons and chlorinated alkanes. Most preferably, the solvent is dichloromethane, as well as dichloroethane and trichloroethane.

The reaction is preferably carried out at mild temperatures. Preferably the cyclisation reaction is performed at a temperature In the range of 20-150° C.

Where a solvent is present, the cyclisation may be performed at reflux temperature, for example, at the reflux temperature of dichloromethane. Optionally the reaction may be carried out below reflux temperature under pressure.

The reaction time may vary from about 2 hours to 12 hours or more and is typically about 2 hours or more. It will be appreciated that reaction conditions may be varied depending upon the individual nature of the substrate and the desired rate of the reaction.

Non-limiting examples of compounds of formula II, which may be described as 5-alkyl-5-hydroxy substituted 1,5-dihydropyrrol-2-ones, that can be synthesised by the method of the invention include:

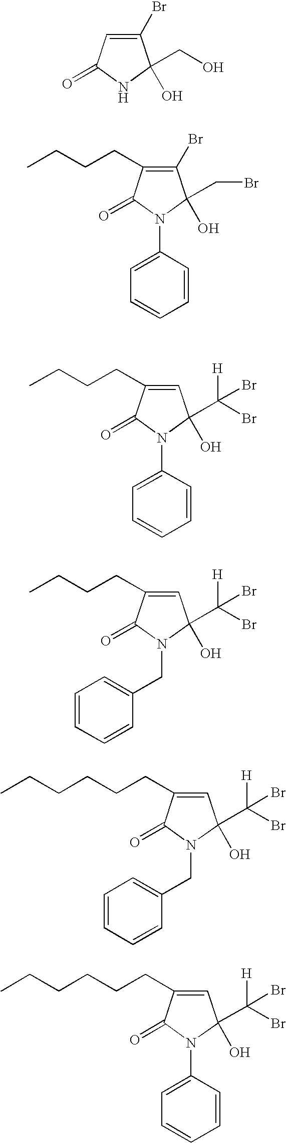

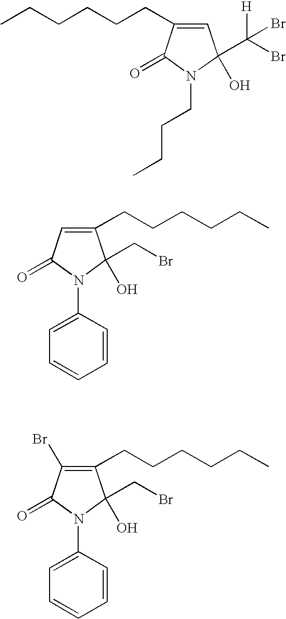

In a second aspect, the present invention provides a compound of formula II: 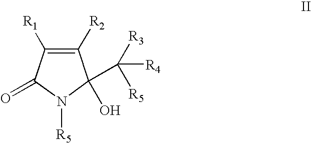
wherein R₁ and R₂ are independently H, halogen, alkyl, substituted or unsubstituted alkoxy, substituted or unsubstituted oxoalkyl, substituted or unsubstituted alkenyl, substituted or unsubstituted aryl or substituted or unsubstituted arylalkyl, optionally interrupted by one or more hetero atoms, straight chain or branched chain, hydrophilic or fluorophilic;
R₃ and R₄ are independently H, halogen, substituted or unsubstituted alkyl, substituted or unsubstituted alkoxy, substituted or unsubstituted aryl or arylalkyl;
R₅ is selected from the group consisting of H, hydroxy, substituted or unsubstituted alkyl, substituted or unsubstituted alkoxy, substituted or unsubstituted oxoalkyl, substituted or unsubstituted alkenyl, substituted or unsubstituted aryl or substituted or unsubstituted arylalkyl, optionally interrupted by one or more hetero atoms, straight chain or branched chain, hydrophilic or fluorophilic, or
forms part of an amino acid, or
is a nucleoside, an oligomer, a polymer, a dendrimer, a substrate or a surface.

Particularly preferred are compounds of formula II in which at least one of R₁, R₂, R₃ and R₄ is halogen.

The inventors have found the 5-alkyl-5-hydroxy substituted 1,5-dihydro pyrrol-2-one of formula II can be dehydrated to yield a range of 5-(halomethylene) 1,5-dihydro-pyrrol-2-one, 5-(dihalomomethylene)-1,5-dihydro pyrrol-2-one.

Accordingly in a third aspect, the present invention provides a method for the dehydration of a compound of formula II above, to prepare a compound of formula III; 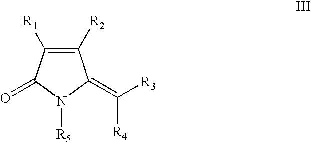
wherein R₁ and R₂ are independently selected from H, halogen, substituted or unsubstituted alkyl, substituted or unsubstituted alkoxy, substituted or unsubstituted oxoalkyl, substituted or unsubstituted alkenyl, substituted or unsubstituted aryl or substituted or unsubstituted arylalkyl, optionally interrupted by one or more hetero atoms, straight chain or branched chain, hydrophilic or fluorophilic;
R₃ and R₄ are independently selected from H, halogen, substituted or unsubstituted alkyl, substituted or unsubstituted alkoxy, substituted or unsubstituted aryl or substituted or unsubstituted arylalkyl; and
R₅ is a defined above,
the method comprising contacting a compound of formula II with a dehydrating agent

Preferably at (east one of R₁, R₂, R₃ and R₄ in formula III is halogen;

Examples of suitable dehydrating agents include phosphorus pentoxide, silica gel, molecular sieves, alumina, acidic resins and polymers, phosphorus oxychloride, acetic anhydride, N,N′-dicyclohexylcarbodiimide (DCC), trifluoroacetic acid, sulfuric acid, trifluoroacetic anhydride, trifluorosulfonic acid anhydride (triflic anhydride).

Preferably dehydration is carried out using phosphorus pentoxide in the presence of a solvent. The solvent may be any suitable solvent. Preferable solvents in the present invention include alkyl acetates, aromatic hydrocarbons, chlorinated alkanes, tetrahydrofuran, diethyl ether, dioxane and C1-C3 acids. More preferably, the solvents are aromatic hydrocarbons and chlorinated alkanes. Most preferably, the solvent is dichloromethane, as well as dichloroethane and trichloroethane.

The reaction is preferably carried out at mild temperatures. Preferably the dehydration reaction is performed at a temperature in the range of from about 20-150° C.

Where a solvent is present, the cyclisation may be performed at reflux temperature of the solvent, for example, at the reflux temperature of dichloromethane.

The reaction time may range from about 2 hours to 12 hours or more and is typically about 2 hours or more. It will be appreciated that reaction conditions may be varied depending on the individual nature of the substrate and the desired rate of the reaction.

Non-limiting examples of furanones (III) that can be synthesised by this procedure are listed below.

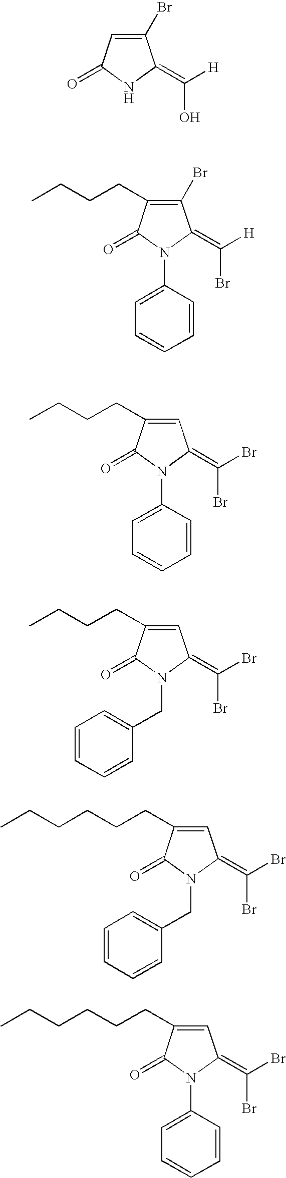

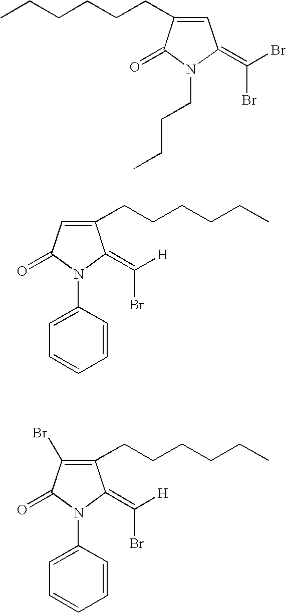

We believe that the 1,5-dihydro-pyrrol-2-ones prepared of formula III are novel compounds.

Thus, in a fourth aspect the present invention provides a compound of formula III: 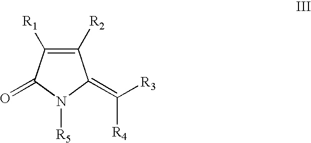
wherein R₁ and R₂ are independently selected from H, halogen, substituted or unsubstituted alkyl, substituted or unsubstituted alkoxy, substituted or unsubstituted oxoalkyl, substituted or unsubstituted alkenyl, substituted or unsubstituted aryl or substituted or unsubstituted arylalkyl, optionally interrupted by one or more hetero atoms, straight-chain or branched chain, hydrophilic or fluorophilic;
R₃ and R₄ are independently selected from H, halogen, substituted or unsubstituted alkyl, substituted or unsubstituted alkoxy, substituted or unsubstituted aryl or arylalkyl; and
R₅ is as defined above.

Preferably at least one of R₁, R₂, R₃ and R₄ is halogen.

Furthermore the present inventors have also found that furanones of formula (I) when treated with certain amines can yield 5-amino substituted or 5-aminomethylene substituted furanones. Alternatively, the compounds of formula I can be treated with an alcohol to yield 5′ alkoxy substituted furanones. For example when 4-bromo-5-bromomethylene-2(5H)-furanone was treated with aniline it gave 4-bromo-5-phenylaminomethylene-2(5H)-furanone in good yields. In contrast, the reaction of 4-bromo-5-bromomethylene-2(5H)-furanone with benzyl amine, gave the corresponding 5-benzylamino-4-bromo-5-bromomethyl-2(5H)-furanone.

Accordingly, in a fifth aspect, the present invention provides a method for the preparation of a compound of formula IV 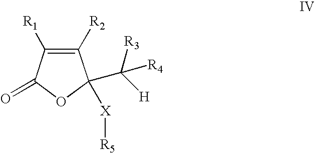
wherein R₁ and R₂ are independently selected from H, halogen, substituted or unsubstituted alkyl, substituted or unsubstituted alkoxy, substituted or unsubstituted oxoalkyl, substituted or unsubstituted alkenyl, substituted or unsubstituted aryl or substituted or unsubstituted arylalkyl, optionally interrupted by one or more hetero atoms, straight chain or branched chain, hydrophilic or fluorophilic;
R₃ and R₄ are independently selected from H, halogen, substituted or unsubstituted alkyl, substituted or unsubstituted alkoxy, substituted or unsubstituted aryl or substituted or unsubstituted arylalkyl;
R₅ is as defined above,
X Is O or NR₆, where R₆ may be R₁,
the method comprising reacting a compound of formula I wherein R₃ is a hydrogen and 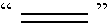
   represents a double bond.

Preferably at least one of R₁, R₂, R₃ and R₄ is halogen. Preferably R₆ is H.

Representative examples of furanones (IV) that can be synthesised by this procedure are listed below.

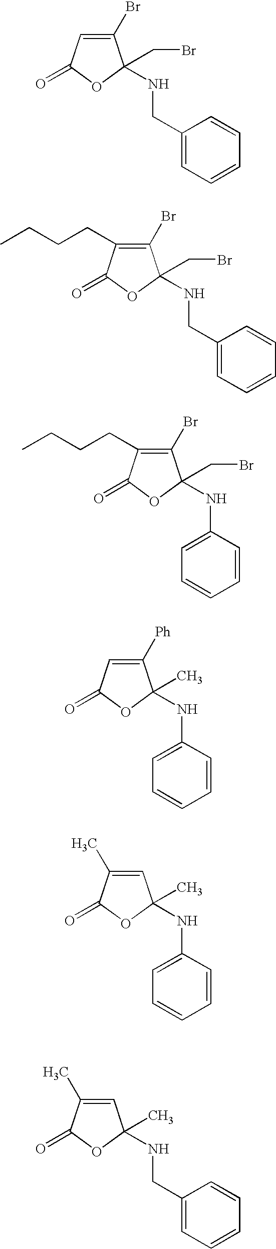

In yet a sixth aspect, the present invention provides a compound of formula IV 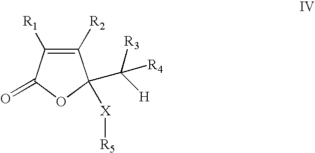
wherein R₁ and R₂ are independently selected from H, halogen, substituted or unsubstituted alkyl, substituted or unsubstituted alkoxy, substituted or unsubstituted oxoalkyl, substituted or unsubstituted alkenyl, substituted or unsubstituted aryl or substituted or unsubstituted arylalkyl, optionally interrupted by one or more hetero atoms, straight chain or branched chain, hydrophilic or fluorophilic;
R₃ and R₄ are independently selected from H, halogen, substituted or unsubstituted alkyl, substituted or unsubstituted alkoxy, substituted or unsubstituted aryl or substituted or unsubstituted arylalkyl; and
R₅ and X are as defined above.

Preferably, at least one of R₁, R₂, R₃ and R₄ is halogen.

Accordingly a seventh aspect, the present invention provides for a method for preparation of a compound of formula V. 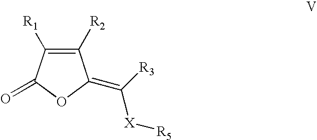
wherein R₁ and R₂ are independently selected from H, halogen, substituted or unsubstituted alkyl, substituted or unsubstituted alkoxy, substituted or unsubstituted oxoalkyl, substituted or unsubstituted alkenyl, substituted or unsubstituted aryl or substituted or unsubstituted arylalkyl, optionally interrupted by one or more hetero atoms, straight chain or branched chain, hydrophilic or fluorophilic;
R₃ is selected from H, halogen, substituted or unsubstituted alkyl substituted or unsubstituted alkoxy, substituted or unsubstituted aryl or arylalkyl; wherein R₅ is H, substituted or unsubstituted alkyl, substituted or unsubstituted alkoxy, substituted or unsubstituted oxoalkyl, substituted or unsubstituted alkenyl, substituted or unsubstituted aryl or substituted or unsubstituted arylalkyl, optionally interrupted by one or more hetero atoms, straight chain or branched chain, hydrophilic or fluorophilic:
X is O or NR₆, where R₆ is as defined above; and
R₅ is as defined above.

Non-limiting examples of furanones of formula (V) that can be synthesised by this procedure are listed below.

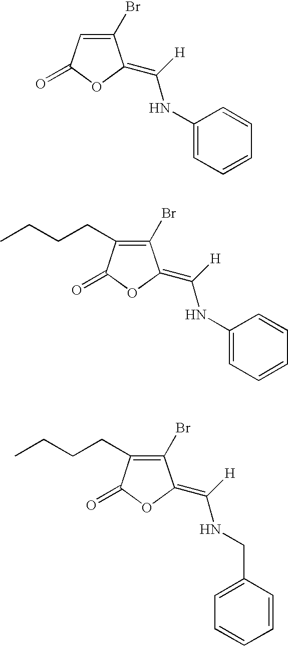

In an eighth aspect, the present invention provides a compound of formula V: 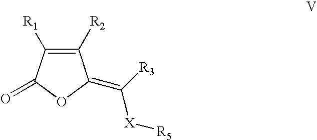
wherein R₁ and R₂ are independently selected from H, halogen, substituted or unsubstituted alkyl, substituted or unsubstituted alkoxy, substituted or unsubstituted oxoalkyl, substituted or unsubstituted alkenyl, substituted or unsubstituted aryl or substituted or unsubstituted arylalkyl, optionally interrupted by one or more hetero atoms, straight chain or branched chain, hydrophilic or fluorophilic;
R₃ is selected from H, halogen, substituted or unsubstituted alkyl, substituted or unsubstituted alkoxy, substituted or unsubstituted aryl or arylalkyl;
X is O or NR₆, where R₆ is as defined above; and
R₅ is as defined above.

In yet a ninth aspect the present invention provides a compound of formula (VI): 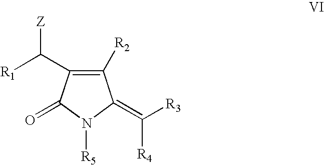
wherein R₁ and R₂ are independently selected from H, halogen, substituted or unsubstituted alkyl, substituted or unsubstituted alkoxy, substituted or unsubstituted oxoalkyl, substituted or unsubstituted alkenyl, substituted or unsubstituted aryl or substituted or unsubstituted arylalkyl, optionally interrupted by one or more hetero atoms, straight chain or branched chain, hydrophilic or fluorophilic;
R₃ and R₄ are independently selected from H, halogen, alkyl, substituted or unsubstituted aryl or arylalkyl; wherein R₅ is H, substituted or unsubstituted alkyl, substituted or unsubstituted alkoxy, substituted or unsubstituted oxoalkyl, substituted or unsubstituted alkenyl, substituted or unsubstituted aryl or substituted or unsubstituted arylalkyl, optionally interrupted by one or more hetero atoms, straight chain or branched chain, hydrophilic or fluorophilic;
R₅ is defined as above; and

Z is selected from the group R₂, halogen, OC(O)R₂, ═O, amine azide, thiol, R₂, mercaptoaryl, arylalkoxy, mercaptoarylalkyl, SC(O)R₂, OS(O)R₂, NHC(O)R₂, ═NR₂ or NHR₂.

The compounds of formula VI may be prepared by functionalizing a fimbrolide of formula (III) wherein, R₁, R₂, R₃ and R₄ are as defined above, with a reagent described in WO 99/54323, (the disclosure of which is incorporated herein by cross-reference).

Reagents for introduction and manipulation of the Z group include halogenating and oxidising agents (N-halosuccinimide, lead tetraacetate, selenium dioxide, Jones reagent), nucleophiles (Including organic metal carboxylates, organic alcohols, dimethyl sulfoxide and organonitriles) and electrophiles including (organic acids, isocyanates, carboxylic or sulfonic acid halides and diethylaminosulfur trifluoride).

Non-limiting examples of furanones of formula (VI) that can be synthesised by this procedure are listed below.

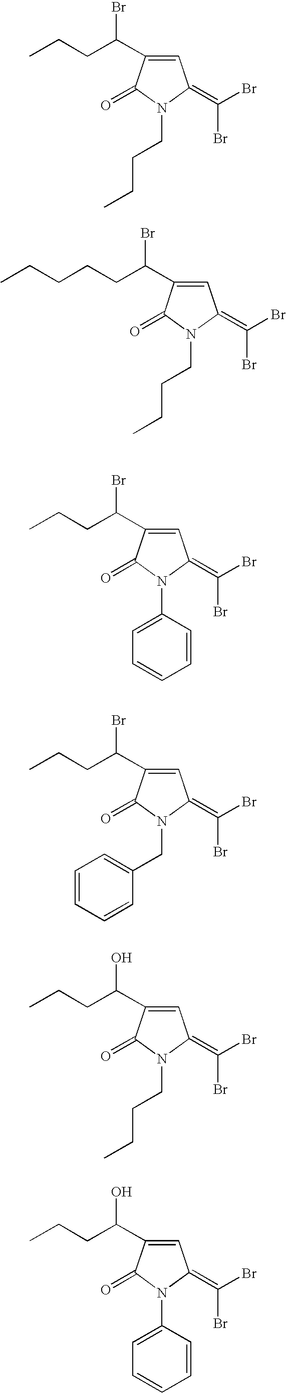

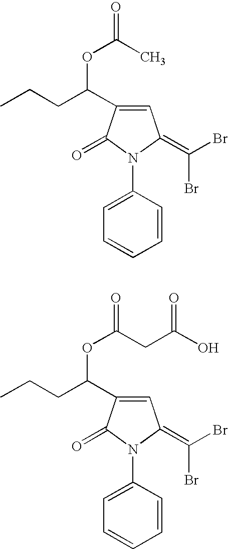

In a tenth aspect, the present invention provides an oligomer or polymer formed by oligomerising or polymerising a compound of formula II-VI, described herein directly or with one or more other monomers.

The one or more other monomer may be any suitable polymerisable copolymer e.g. acrylate ester such as alkyl, hydroxyalkyl, aminoalkyl, or substituted substituted or unsubstituted aryl acrylates or methacrylates; crotonates, substituted or unsubstituted acrylonitriles, vinyl alcohols or acetates, styrene and siloxanes.

R₅ may be a residue of a natural or synthetic compound. R₅ may be a biological or non-biological compound. For example, R₅ may be a coenzyme or cofactor. R₅ may be an oligomer or a polymer, which may be biological or synthetic. For example, the oligomer or polymer may be a peptide or polyamide. The polymer may be a protein, for example, an enzyme or a receptor. R₅ may be an oligomer or polymer comprising nucleic acid residues. The polymer may be a polynucleotide, for example, DNA or RNA. R₅ may form part of or be bonded to a nucleoside. The nucleoside may be a D or L-nucleoside. R5 may be linked to a sugar moiety of the nucleoside.

R₅ may be a surface or substrate with which the nitrogen atom of is associated. The association may be chemical bonding, for example covalent bonding. The surface or substrate may be biological or synthetic. Alternatively, the association may be by means of adsorption. Methods for forming such associations are described in more detail below.

R₅ may also be a dendrimer. A review of dendrimers is provided in Klajnert, B. and Bryszewska, M. (2001) Dendrimers: properties and applications, Acta Biochemica Polonica Vol. 48 No. 1/2001, the disclosure of which is incorporated herein by reference. A plurality of compounds in accordance with the invention may be carried by the dendrimer.

The compound may be immobilised directly onto at least part of the surface of the material of the substrate or via one or more intermediate layers interposed between the substrate material and the immobilised layer. The intermediate layer (s) may be bonding layer(s).

The substrate may be shaped or non-shaped. The substrate may be solid, semi-solid or flexible. The substrate may be a woven or non-woven film or sheet. The substrate may be a natural or synthetic filament or fibre. The substrate may be a natural material, for example, a plant seed. The material from which the substrate is formed may be selected to suit the particular application. For example, in the case of a shaped biomedical device the material may meet other specifications of the application, such as mechanical and optical properties.

The substrate may be a shaped article including, but are not limited, medical devices, for example, implantable biomedical devices such as urinary catheters, percutaneous access catheters, stents, as well as non-implantable devices such as contact lenses, contact lens storage cases, and the like.

The material from which the article is formed can be a metal, a ceramic, a solid synthetic polymer, or a solid natural polymer, for example a solid biopolymer. Examples of useful materials for this invention are titanium, hydroxyapatite, polyethylene (which are useful materials for orthopaedic implants), polyurethanes, organosiloxane polymers, perfluorinated polymers (which are useful materials for instance for catheters, soft tissue augmentation, and blood contacting devices such as heart valves), acrylic hydrogel polymers and siloxane hydrogel polymers (for instance for contact lens and intraocular lens applications), and the like, and any combination thereof. The surfaces of these materials can be chemically inert or contain reactive functional groups.

Further examples of substrates include archival documents, antiques and art, rare and valuable seeds intended for storage (e.g. seed banks of conservation groups), etc in which case the substrate may be paper, material or other natural or synthetic material.

The substrate may be a shell fish or aquaculture apparatus, for example, that described in PCT/AU98/00508, the disclosure of which is incorporated herein by reference.

As mentioned above, R₅ may be associated with a surface of substrate. If necessary, the surface of the substrate may be optionally treated at least in part to activate the surface, to which the compounds of the present invention may be reacted to immobilise the compound.

Reference to at least part of the surface of the substrate includes a surface of one or more intermediate layers applied to the substrate.

The compounds may be immobilised on the substrate surface by any suitable technique. Immobilization may be by covalent or non covalent means. Preferably, the compounds are immobilized on the substrate surface by means of covalent bonds.

The immobilization of furanone compounds on to the substrate prevents their loss from the surface, thus ensuring long-lasting antimicrobial action.

The association between the compounds of the invention and the substrate may be characterised by the formula: X—Y-Z where X is a substrate. Y is an optional chemical linking moiety and Z is a compound in accordance with the present invention. The linking moiety, if present, may be a homobifunctional or heterobifunctional linking moiety, Y may be a simple component (eg a short molecule) or it may comprise a plurality of units or components that may be the same of different. Y may comprise a number of components or units that may be “built up” in a stepwise fashion.

The formation of a covalent interfacial linkage is much preferable to an ionic bond since in biological media where the salt content is such that ionic bonds are interfered with and ironically attached molecules can be displaced from a surface.

In the context of substrates that are medical devices, covalent anchoring of the compound(s) also serves to eliminate concerns regarding possible deleterious effect that compounds might cause at sites distant from the device, such as in the liver, brain, or kidney tissues of a living human organism. In medical applications it is important to anchor the furanone compound (s) via an interfacial covalent bond that is not subject to cleavage in the host environment that the biomedical device is to be placed in.

Methods for the covalent immobilization of organic molecules onto solid surfaces are well known to those skilled in the art. Interfacial reactions leading to the formation of covalent interfacial bonds are derived from well known organic-synthetic reactions. The choice of immobilization reaction depends on both the nature of the substrate material and the chemical composition of the furanone derivative (s) that are desired for a particular application.

For example, a compound that contains a hydroxyl group in a side chain distal to the ring system, can be linked covalently onto surfaces using epoxide chemistry analogous to the reaction pathway described for the immobilization of polysaccharides onto epoxidated surfaces in Li et al., Surface Modification of Polymeric Biomaterials (B D Ratner and D G Castner, Eds), Plenum Press, NY, 1996 pages 165-173 (the disclosure of which is incorporated herein in its entirety), through isocyanate groups attached to the surface to produce stable urethane linkages through thermal processes, or through carboxylic acid groups or their equivalents, such as acid chlorides, on the surface to produce ester linkages. A compound that contains an aldehyde group can be linked onto surface amine groups using a reductive animation reaction. A compound that contains a carboxylic acid group can be linked onto surface amine groups using carbodiimide chemistry.

Interfacial coupling reactions must of course be selected not only for their ability to achieve the desired covalent linkage but also for avoidance of adverse effects on the furanone compound (s) to be attached. Particularly, the furanone ring system tends to be labile to alkaline conditions. Such limitations are well known to those skilled in the art. Among the many possible interfacial coupling reactions known in the art, there is sufficient scope for selection of reactions that proceed in a suitable pH range and with furanones substituted with various functional groups in various positions.

Some solid substrate materials possess reactive surface chemical groups that can undergo chemical reactions with a partner group on a compound and thereby form a covalent interfacial linkage directly.

Alternatively, in situ covalent linkage can be made directly through the addition of a doubly functionalised linker molecule to the active surface in the presence of an appropriate compound, or stepwise by sequential addition of doubly functionalised linker molecules and then an appropriate compound. It is not always possible to immobilize furanone compounds directly onto solid substrate materials; in these cases, surface activation or one or more interfacial bonding layer (s) is used to effect covalent immobilization of the compounds. Such surface activation is essential when immobilizing compounds onto polymeric materials such as fluoropolymers and polyolefins.

Surface activation of solid substrate materials can be achieved in a number of ways. Examples are corona discharge treatment or low pressure plasma treatment of polymers. These methods are well known to introduce a variety of functional groups onto polymeric surfaces.

An alternative approach is to provide an interfacial bonding layer interspersed between the solid substrate material or medical device and the compound layer. The application of a thin Interfacial bonding layer can be done using methods such as dip coating, spin coating, or plasma polymerization. The chemistry of the bonding layer is selected such that appropriate reactive chemical groups are provided on the surface of this layer, groups that then are accessible for reaction with compound of the invention.

Particularly versatile is the subsequent application of multiple thin interfacial bonding layers; this method can provide a very wide range of desired chemical groups on the surface for the immobilization of a wide range of functionalized furanones and enables usage of compounds optimized for their biological efficacy.

By providing a thin, surface-coated layer of compounds, the optical quality of antibacterial devices of this invention is not reduced, which makes the invention applicable to transparent ophthalmic devices such as contact lenses and intraocular lenses.

The present invention provides thin surface coatings that provide antimicrobial properties and/or antifungal properties to solid materials onto which the coatings have been applied. More particularly, the coatings may be designed to reduce or prevent colonization of biomedical devices by bacteria that cause adverse effects on the health of human users of biomedical devices when such devices are colonized by bacteria.

The active antibacterial layer comprises one or a plurality of furanone compounds selected for both their antibacterial activity and absence of cytotoxicity as well as any other adverse biomedical effect on the host environment that the coated device contacts.

In an eleventh aspect, the present invention provides incorporation of compounds produced by the methods according to the first, third, fifth, seventh, ninth, or tenth aspects either in surface coatings or polymers through any part of the molecule, for example, newly introduced functionality on the alkyl chain or the alkyl chain or the halomethylene functionality itself via direct polymerisation or copolymerisation with suitable monomers.

In an twelfth aspect, the present invention provides a compound produced by the method according to the first, third, fifth, seventh, ninth, or eleventh aspects of the present invention.

In a thirteenth aspect, the present invention provides the use of a compound produced according to the present invention. The present inventors have found that many of the 1,5-dihydropyrrol-2-one derivatives and furanones having the formula (II), (III), (IV), (V) and (VI) have antimicrobial and/or antifouling properties. Accordingly, the fimbrolide derivatives are suitable for use as antimicrobial and/or antifouling agents.

Thus in a fourteenth aspect, the present invention provides methods of use of compounds of formula (II), (III), (IV), (V) and (VI) in medical, scientific and/or biological applications.

For these and other applications, the compounds of the present invention may be formulated as a composition.

In a fifteenth aspect, the present invention provides a composition comprising at least one compound of formula (II), (III), (IV), (V) or (VI).

The compositions of the third aspect of the invention may be in any suitable form. The composition may include a carrier or diluent. The carrier may be liquid or solid. For example, the compositions may be in the form of a solution or suspension of at least one of the compounds in a liquid. The liquid may be an aqueous solvent or a non-aqueous solvent. The liquid may consist of or comprise a one or more organic solvents. The liquid may be an ionic liquid. Particular examples of carrier or diluents include, but are not limited to, water, polyethylene glycol, propylene glycol, cyclodextrin and derivatives thereof.

The composition may be formulated for delivery in an aerosol or powder form.

The composition may include organic or inorganic polymeric substances. For example, the compound of the invention may be admixed with a polymer or bound to, or adsorbed on to, a polymer.

When the composition is to be formulated as a disinfectant or cleaning formulation, the composition may include conventional additives used in such formulations. Non-limiting examples of the physical form of the formulations include powders, solutions, suspensions, dispersions, emulsions and gels.

Formulations for pharmaceutical uses may incorporate pharmaceutically acceptable carriers, diluents and excipients known to those skilled in the art The compositions make be formulated for parenteral or non-parenteral administration. The composition of the invention may be formulated for methods of introduction including, but not limited to, topical, intradermal, intramuscular, intraperitoneal, intravenous, subcutaneous, intranasal, epidural, ophthalmic, and oral mutes. It may be formulated for administration by any convenient route, for example by infusion or bolus injection, by absorption through epithelial or mucocutaneous linings (e.g., oral mucosa, rectal and intestinal mucosa, etc.) and may be administered together with other biologically active agents. Administration may be localized or systemic. The composition may be formulated for intraventricular and intrathecal injection. Pulmonary administration can also be employed, e.g., by use of an inhaler or nebulizer, and formulation with an aerosolizing agent.

In certain preferred embodiments the composition further comprises other active agents such as antibiotics and cleaning agents.

In a sixteenth aspect, the present invention provides a method of treating an infection in a human or animal subject the method comprising administration to the subject of an effective amount of the compound of the invention.

The treatment may therapeutic and/or prophylactic.

The compounds of the present invention can act as quorum sensing inhibitors and therefore find use in any application where such as effect is desired. For example, the compounds of the present invention may have use in preventing the establishment and expression of virulence by microorganisms through the inhibition of quorum sensing systems and/or other extracellular systems (eg see, International patent application No. PCT/AU01/01621, the disclosure of which is incorporated herein in its entirety).

The present invention is suitable for biofilms originating from a single type of organism and for mixed biofilms. By “mixed biofilms” is meant biofilms created by more than one type of microorganism. Most preferably, it is envisioned that biofilms will be created by at least two organisms from the group consisting of bacteria, algae, fungi, and protozoa.

The effects of treating biofilms with homoserine lactones have been demonstrated with Pseudomonas aeruginosa. The HSLs have generally been isolated from a wide range of bacteria known to produce biofilms. Among these are the enterobacteria. The presence of the HSLs in a wide range of bacteria indicates that the compounds of the present invention can be used to effectively treat not only Pseudomonas sp. biofilms but also mixed biofilms containing Pseudomonas sp. and biofilms composed of bacteria other than Pseudomonas aeruginosa.

The following is a list of groups of Gram-Negative bacteria that have members which use homoserine lactones for cell-cell communication: anaerobic Gram Negative Straight, Curved and Helical Rods; Bacteroidaceae; The Rickettsias and Chlamydias; Dissimilatory Sulfate—or Sulfur-Reducing Bacteria; the Mycoplasmas; The mycobacteria; Budding and/or Appendaged Bacteria; Sheathed Bacteria; Nocardioforms, and Actinomycetes, for example. See Bergey's Manual of Systematic Bacteriology, First Ed., John G. Holt, Editor in Chief (1984), incorporated herein by reference.

The method of the sixteenth aspect may be used to treat an infection or condition in a subject that is characterised by biofilm formation. Non-limiting examples of human infections involving biofilms include dental caries, periodontitis, otitis media, muscular skeletal infections, necrotising fascitis, biliary tract infection, osteomyelitis, bacterial prostatitis, native valve endocarditis, cystic fibrosis pneumonia, meloidosis, and nosocomial infections such as ICU pneumonia, sutures, exit sites, arteriovenous sites, scleral buckles, contact lenses, urinary catheter cystitis, peritoneal dialysis (CAPD) peritonitis, IUDs, endotracheal tubes, Hickman catheters, central venous catheters, mechanical heart valves, vascular grafts, biliary stent blockage, and orthopedic devices, penile prostheses. Further applications are described in Costerton J et al, (1999) Vol. 284, Science pp 1318-1322 and Costerton J and Steward, (2001) Battling Biofilms, Scientific American pp 75-81, the disclosures of which are incorporated herein by reference.

Other locations in which biofilms may form included drinking water pipes, which may lead to corrosion or disease, household drains, dental plaque which may lead to gum disease and cavities, which may lead to gun disease or cavities, contact lenses which may lead to eye infections, ears which may lead to chronic infection and lungs which may lead to pneumonia.

The condition may be cystic fibrosis. The infection may be that resulting from a skin infection, burn infection and/or wound infection. The method and composition of the invention may be particularly suitable for the treatment of infection in immuno compromised individuals.

In yet a seventeenth aspect, the present invention provides a method for treating biofilm formation on a surface by contacting the surface with a compound in accordance with the present invention.

The term “surface” as used herein relates to any surface which may be covered by a biofilm layer. The surface may be a biological (eg tissue, membrane, skin etc) or non-biological surface.

The surface may be that of a natural surface, for example, plant seed, wood, fibre etc.

The surface or substrate may be any hard surface such as metal, organic and inorganic polymer surface, natural and synthetic elastomers, board, glass, wood, paper, concrete, rock, marble, gypsum and ceramic materials which optionally are coated, eg with paint, enamel etc; or any soft surface such as fibres of any kind (yams, textiles, vegetable fibres, rock wool, hair etc.); or porous surfaces; skin (human or animal); keratinous materials (nails etc.). The hard surface can be present in process equipment or components of cooling equipment, for example, a cooling tower, a water treatment plant, a dairy, a food processing plant, a chemical or pharmaceutical process plant. The porous surface can be present in a filter, eg. a membrane filter.

Particular examples of surfaces that may be treated in accordance with the invention include, but are not limited to, toilet bowls, bathtubs, drains, highchairs, counter tops, vegetables, meat processing rooms, butcher shops, food preparation areas, air ducts, air-conditioners, carpets, paper or woven product treatment, nappies (diapers), personal hygiene products (eg sanitary napkins) and washing machines. The cleaning composition may be in the form of a toilet drop-in or spray-on devices for prevention and removal of soil and under rim cleaner for toilets. The compositions and methods of the present invention also have applications in cleaning of Industrial surfaces such as floors, benches, walls and the like and these and other surfaces in medical establishments such as hospitals (eg surfaces in operating theatres), veterinary hospitals, and in mortuaries and funeral pariours.

A compound of the invention may be incorporated into epidermal bandages and lotions. Alternatively, the compounds of the invention may be incorporated into cosmetic formulations, for example, after shave lotions.

Compositions of the present invention may be in the form of an aqueous solution or suspension containing a cleaning-effective amount of the active compound described above. The cleaning composition may be in the form of a spray, a dispensable liquid, or a toilet tank drop-in, under-rim product for prevention, removal and cleaning of toilets and other wet or intermittently wet surfaces in domestic or industrial environments.

The compositions of the present invention may additionally comprise a surfactant selected from the group consisting of anionic, non-ionic, amphoteric, biological surfactants and mixtures thereof. Most preferably, the surfactant is sodium dodecyl sulfate.

One or more adjuvant compounds may be added to the cleaning solution of the present invention. They may be selected from one or more of biocides, fungicides, antibiotics, and mixtures thereof to affect planktonics. pH regulators, perfumes, dyes or colorants may also be added.

By “cleaning-effective” amount of active compound, it is meant an amount of the compound which is necessary to remove at least 10% of bacteria from a biofilm as determined by a reduction in numbers of bacteria within the biofilm when compared with a biofilm not exposed to the active compound.

The cleaning methods of the present invention are suitable for cleaning surfaces. They may be used to treat hard, rigid surfaces such as drain pipes, glazed ceramic, porcelain, glass, metal, wood, chrome, plastic, vinyl and formica or soft flexible surfaces such as shower curtains, upholstery, laundry and carpeting. It is also envisioned that both woven and non woven and porous and non-porous surfaces would be suitable.

In other embodiments of the present invention, the composition of the invention may be formulated as a dentifrice, a mouthwash or a composition for the treatment of dental caries. The composition may be formulated for acne treatment or cleaning and disinfecting contact lenses (eg as a saline solution).

The method of the invention may be used to treat medical devices.

In yet a further aspect, the present invention extend to a medical device having a least one surface associated with a compound(s) in accordance with the present invention.

The method of the invention may be used to treat implanted devices that are permanent such as an artificial heart valve or hip joint, and those that are not permanent such as indwelling catheters, pacemakers, surgical pins etc. The method may further be used in situations involving bacterial infection of a host, either human or animal, for example in a topical dressing for burn patients. An example of such a situation would be the infection by P. aeruginosa of superficial wounds such as are found in burn patients or in the lung of a cystic fibrosis patient.

In other forms, the present invention can be used to treat integrated circuits, circuit boards or other electronic or microelectronic devices.

In yet another aspect, the present invention provides a method for the inhibition of a biological pathway is a cell, the method comprising administering to the cell a compound in accordance with the present invention.

Terminology

The term “alkyl” is taken to mean both straight chain alkyl groups such as methyl, ethyl, propyl, isopropyl, butyl, isobutyl, sec-butyl, tertiary butyl, and the like. Preferably the alkyl group is a lower alkyl of 1 to 6 carbon atoms. The alkyl group may optionally be substituted by one or more groups selected from alkyl, cycloalkyl, alkenyl, alkynyl, halo, carboxyl, haloalkyl, haloalkynyl, hydroxy, substituted or unsubstituted alkoxy, alkenyloxy, haloalkoxy, haloalkenyloxy, nitro, amino, nitroalkyl, nitroalkenyl, nitroalkynyl, nitroheterocyclyl, alkylamino, dialkylamino, alkenylamine, alkynylamino, acyl, alkenoyl, alkynoyl, acylamino, diacylamino, acyloxy, alkylsulfonyloxy, heterocyclyl, heterocycloxy, heterocyclamino, haloheterocyclyl, alkylsulfenyl, alkylcarbonyloxy, alkylthio, acylthio, phosphorus-containing groups such as phosphono and phosphinyl.

The term “alkoxy” denotes straight chain or branched alkyloxy, preferably C₁₋₁₀ alkoxy. Examples include methoxy, ethoxy, n-propoxy, isopropoxy and the different butoxy Isomers.

The term “alkenyl” includes groups formed from straight chain, branched or mono or polycyclic alkenes and polyene. Substituents include mono or poly-unsaturated alkyl or cycloalkyl groups as previously defined, preferably C₂₋₁₀ alkenyl. Examples of alkenyl include vinyl, allyl, 1-methylvinyl, butenyl, iso-butenyl, 3-methyl-2-butenyl, 1-pentenyl, cyclopentenyl, 1-methyl-cyclopentenyl, 1-hexenyl, 3-hexenyl, cyclohexenyl, 1-heptenyl, 3-heptenyl, 1-octenyl, cyclooctenyl, 1-nonenyl, 2-nonenyl, 3-nonenyl, 1-decenyl, 3-decenyl, 1,3 butadienyl, 1,4-pentadienyl, 1,3-cyclopentadienyl, 1,3-hexadienyl, 1,4-hexadienyl, 1,3-cyclohexadienyl, 1,4-cyclohexadienyl, 1,3-cycloheptadienyl, 1,3,5-cycloheptatrienyl, or 1,3,5,7-cyclooctatetraenyl.

The term “halogen” includes fluorine, chlorine, bromine or iodine, preferably bromine or fluorine.

The term “heteroatoms” denotes O, N, S or Si.

The term “acyl” used either alone or in compound words such as “acyloxy”, “acylthio”, “acylamino or diacylamino” denotes an alkanoyl, aroyl, heteroyl, carbamoyl, alkoxycarbonyl, alkanesulfonyl, arysulfonyl, and is preferably a C₁₋₁₀ alkanoyl. Examples of acyl include carbamoyl; straight chain or branched alkanoyl, such as formyl, acetyl, propanoyl, butanoyl, 2-methylpropanoyl, pentanoyl, 2,2-dimethylpropanoyl, hexanoyl, heptanoyl, octanoyl, nonanoyl, decanoyl; alkoxycarbonyl, such as methoxycarbonyl, ethoxycarbonyl, t-butoxycarbonyl, t-pentyloxycarbonyl or heptyloxycarbonyl; cycloalkanecarbonyl such as cyclopropanecarbonyl cyclobutanecarbonyl, cyclopentanecarbonyl or cyclohexanecarbonyl; alkanesulfonyl, such as methanesulfonyl or ethanesulfonyl; alkoxysulfonyl, such as methoxysulfonyl or ethoxysulfonyl; heterocycloalkanecarbonyl; heterocyclyoalkanoyl, such as pyrrolidinylacetyl, pyrrolidinylpropanoyl, pyrrolinylacetyl, pyrrolylacetyl, pyrrolidinylbutanoyl, pyrrolidinylpentanoyl, pyrrolidinylhexanoyl or thiazolidinylacetyl; heterocyclylalkenoyl, such as heterocyclylpropenoyl, heterocyclylbutenoyl, heterocyclylpentenoyl or heterocyclylhexenoyl; or heterocyclylglyoxyloyl, such as, thiazolidinylglyoxyloyl or pyrrolidinylglyoxyloyl.

The term “aryl” refers to aryl groups having 6 through 10 carbon atoms and includes, for example, phenyl, naphthyl, indenyl. Typically the aryl group will be phenyl or naphthyl as compounds having such groups are more readily available commercially than other aryl compounds.

The term “substituted aryl” refers to aryl groups having 1 through 3 substituents independently selected from the group of lower alkyl, lower substituted or unsubstituted alkoxy, halonitro, or haloalkyl having 1 through 3 carbon atoms and 1 through 3 halo atoms. Typical substituted aryl groups include, for example, 2-fluorophenyl, 2-chlorophenyl, 2,6-dimethylphenyl, 4-fluorophenyl, 2-methylphenyl, 2-chloro, 3-chloromethylphenyl, 2-nitro, 5-methylphenyl, 2,6-dichlorophenyl, 3-trifluoromethylphenyl, 2-methoxyphenyl, 2-bromonaphth-1-yl, 3-methoxyinden-1-yl, and the like.

Carboxyaryl eg carboxy phenyl, aminoaryl eg aminophenyl

The term “fluorophilic” is used to indicate the highly attractive interactions between certain groups, such as highly fluorinated alkyl groups of C4-C10 chain length, towards perfluoroalkanes and perfluoroalkane polymers.

The term “amino acid” as used herein includes any compound having at least one amino group and at least one carboxyl group. The amino acid may be a naturally occurring amino acid or it may be a non-naturally occurring amino acid.

The amines used in this invention may be soluble in the reaction medium or insoluble in the reaction medium. Examples of soluble amines include ammonia, alkyl-, aryl-, arylalkyl-, and heterocyclic amines.

Examples of insoluble amines include basic amine resins and amine containing biological and synthetic polymers.

The term “optionally substituted” includes, but is not limited to such groups as halogen; hydroxy; hydroxy substituted alkyl; substituted or unsubstituted S(O)ₘ alkyl or S(O)ₘ aryl wherein m is 0, 1 or 2, such as methyl thio, methylsulfinyl or methyl sulfonyl; amino, mono and di-substituted amino; alkyl, cycloalkyl, or cycloalkyl alkyl group; halosubstituted alkyl, such as CF₃; an optionally substituted aryl, optionally substituted arylalkyl, such as benzyl or phenethyl, wherein these aryl moieties may also be substituted one to two times by halogen; hydroxy; hydroxy substituted alkyl; alkoxy; S(O)m alkyl; amino, mono and di-alkyl substituted amino, substituted or unsubstituted alkyl SiO— for example (CH₃)₃SiO—.

The term “Medical devices” as used herein includes disposable or permanent catheters, (e.g., central venous catheters, dialysis catheters, long-term tunneled central venous catheters, short-term central venous catheters, peripherally inserted central catheters, peripheral venous catheters, pulmonary artery Swan-Ganz catheters, urinary catheters, and peritoneal catheters), long-term urinary devices, tissue bonding urinary devices, vascular grafts, vascular catheter ports, wound drain tubes, ventricular catheters, hydrocephalus shunts heart valves, heart assist devices (e.g., left ventricular assist devices), pacemaker capsules, incontinence devices, penile implants, small or temporary joint replacements, urinary dilator, cannulas, elastomers, hydrogels, surgical instruments, dental instruments, tubings, such as intravenous tubes, breathing tubes, dental water lines, dental drain tubes, and feeding tubes, fabrics, paper, indicator strips (e.g., paper indicator strips or plastic indicator strips), adhesives (e.g., hydrogel adhesives, hot-melt adhesives, or solvent-based adhesives), bandages, orthopedic implants, and any other device used in the medical field. “Medical devices” also include any device which may be inserted or implanted into a human being or other animal, or placed at the insertion or implantation site such as the skin near the insertion or implantation site, and which include at least one surface which is susceptible to colonization by biofilm embedded microorganisms. Medical devices also include any other surface which may be desired or necessary to prevent biofilm embedded microorganisms from growing or proliferating on at least one surface of the medical device, or to remove or clean biofilm embedded microorganisms from the at least one surface of the medical device, such as the surfaces of equipment in operating rooms, emergency rooms, hospital rooms, clinics, and bathrooms. In one specific embodiment, the biofilm penetrating composition is integrated into an adhesive, such as tape, thereby providing an adhesive which may prevent growth or proliferation of biofilm embedded microorganisms on at least one surface of the adhesive.

Implantable medical devices include orthopedic implants. Insertable medical devices include catheters and shunts which. The medical devices may be formed of any suitable metallic materials or non-metallic materials known to persons skilled in the art. Examples of metallic materials include, but are not limited to, tivanium, titanium, and stainless steel, and derivatives or combinations thereof. Examples of non-metallic materials include, but are not limited to, thermoplastic or polymeric materials such as rubber, plastic, polyesters, polyethylene, polyurethane, silicone, Gortex™ (polytetrafluoroethylene), Dacron™ (polyethylene tetraphthalate), Teflon (polytetrafluoroethylene), latex, elastomers and Dacron™ sealed with gelatin, collagen or albumin, and derivatives or combinations thereof.

The present invention also extends to a method of regulating a cells characterised by AHL-mediated quorum sensing or an Al-2 pathway comprising contacting the cells with a compound in accordance with the present invention.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Reference is made to patent applications PCT/AU01/01621 PCT/AU02100797, PCT/AU99/00284, PCT/AU99/00285, PCT/AU00/01553, PCT/AU01/00296, PCT/AU01/00295, PCT/AU 01100407. PCT/AU89/00508 and POT/AU01/00781 which relate to furanones and analogues and to uses of these compounds and the entire disclosures of which are incorporated herein by reference.

### MODES FOR CARRYING OUT THE INVENTION

The invention is further described in and illustrated by the following examples. The examples are not to be construed as limiting the invention in any way.

Experimental Details

General. Melting points are uncorrected. Microanalyses were performed by Dr H. P. Pham of The University of New South Wales Microanalytical Laboratory. ¹H NMR spectra were obtained in CDCl₃ on a Bruker AC300F (300 MHz) or a Bruker DMX500 (500 MHz) spectrometer. ¹³C NMR were obtained in the same solvent on a Bruker AC300F (75.5 MHz) or a Bruker DMX500 (125.8 MHz) spectrometer. Chemical shifts were measured on the δ scale internally referenced to the solvent peaks: CDCl₃ (δ 7.26, δ 77.04). Ultraviolet spectra were measured on an Hitachi U-3200 spectrophotometer and refer to solutions in absolute MeOH. Infrared spectra were recorded on a Perkin-Elmer 298 or a Perkin-Elmer 580B spectrophotometer and refer to paraffin mulls. The electron impact mass spectra were recorded on an VG Quattro mass spectrometer at 70 eV ionisation voltage and 200° C. ion source temperature. FAB spectra were recorded on an AutoSpecQ mass spectrometer. Column chromatography was carried out using Merck silica gel 60H (Art. 7736), whilst preparative thin layer chromatography was performed on 2 mm plates using Merck silica gel 60GF₂₅₄ (Art. 7730).

### 3-Butyl-5-dibromomethyl-5-hydroxy-1-phenyl-1,5-dihydropyrrol-2-one

A solution of 3-butyl-5-dibromomethylene-2(5H)-furanone (0.20 g; 0.65 mmol) in aniline (5 ml) was allowed to stand at room temperature for 24 h. The mixture was diluted with dichloromethane (25 ml) and washed with aqueous hydrochloric acid (2M, 20 ml). The organic phase was dried over sodium sulfate and evaporated to yield a yellow viscous oil (0.30 g). The crude product was chromatographed on silica using dichloromethane/ethylacetate (19:1; v:v) as the eluent. The major product, a pale yellow band, was collected and recrystallised from light petroleum to yield 3-butyl-5-dibromomethyl-5-hydroxy-1-phenyl-1,5-dihydropyrrol-2-one as colourless prisms (0.24 g, 92%), m.p. 96-98° C. νₘₐₓ 3211, 2957, 1679, 1597, 1500, 1417, 1117, 1058, 760, 698 cm⁻¹. λₘₐₓ 263 nm (εₘₐₓ 2,955), 202 (2,464). ¹H n.m.r. δ (CDCl₃): 7.54-7.37, m, Ph; 6.82, 1H, s, C4-H, 5.56, 1H, s, —CHBr₂; 3.42, s, C5-OH, 2.43-2.41, m, 2H, CH₂; 1.64-0.97, m, C3-chain. ¹³C n.m.r. δ (CDCl₃): 13.7, 14.0, 22.3, 46.6, 92.2, 126.7, 127.4, 129.0, 134.0, 135.5, 136.0, 144.3, 169.0.

### 5-Dibromomethyl-3-hexyl-5-hydroxy-1-phenyl-1,5-dihydropyrrol-2-one

A mixture of 3-hexyl-5-dibromomethylene-2(5H)-furanone (0.40 g, 1.18 mmol) and aniline (1 ml) in ethanol (6 ml) was refluxed for 3 h. The solvent was evaporated off and the residue extracted with dichloromethane (25 ml). The organic phase was washed with aqueous hydrochloric acid (2M, 2×20 ml), dried over sodium sulfate and evaporated to yield a semi-solid (0.39 g). The crude product was chromatographed on silica using dichloromethane/ethylacetate (19:1; v:v) as the eluent. The major product, a pale yellow band, was collected and recrystallised from light petroleum to yield 5-dibromomethyl-3-hexyl-5-hydroxy-1-phenyl-1,5-dihydropyrrol-2-one as a semi-crystalline-solid (23%), m.p. 43-45° (Found (HRESMS) 451.982217. C₁₇H₂₁Br₂NO₂Na⁺ (⁷⁹Br) requires 451.983106). νₘₐₓ: 3186, 2926, 1680, 1659, 1492, 1372, 1095, 1059, 897, 850, 766, 747, 699, 671 cm⁻¹: λₘₐₓ; 261 nm (⊖ₘₐₓ 4051), 206 (26.550). ¹H n.m.r. δ (CDCl₃): 7.5-7.25, m, 5H, Ph; 6.8, s, 1H, C4-H; 5.5, s, 1H, —CHBr₂; 3.77, brs, 1H, C5-OH; 2.44-2.34, m, 2H, CH₂; 2.03-0.91, 11H, C3-chain. ¹³C n.m.r. δ (CDCl₃): 14.2, 25.3, 29.0, 31.0, 46.6, 92.0, 104.8, 126.7, 127.0, 136.0, 144.0, 169.0, 172.0.

### 1-Benzyl-3-butyl-5-dibromomethyl-5-hydroxy-1,5-dihydropyrrol-2-one

A solution of 3-butyl-6-dibromomethylene-2(5H)-furanone (1.03 g: 3.32 mmol) in benzyl amine (2 ml) was allowed to stand at room temperature for 1 h during which time the reaction mixture solidified. The solid was dissolved in dichloromethane (25 ml) and washed with aqueous hydrochloric acid (2M, 20 ml). The organic phase was dried over sodium sulfate and evaporated to yield a yellow viscous oil. The crude product was triturated with light petroleum to yield a white solid (1.0 g; 74%) which was recrystallised from light petroleum to yield 1-benzyl-3-butyl-5-dibromomethyl-5-hydroxy-1,5-dihydropyrrol-2-one as colourless needles, m.p. 92-93° C. (Found (HRESMS) m/z-479.974243. C₁₈H₂₁Br₂NO₃Na⁺ (⁷⁹Br) requires 479.978123). νₘₐₓ: 2987, 2953, 2920, 1677, 1650, 1449, 1424, 1069 cm¹. λₘₐₓ; 207 (88,250). ¹H n.m.r. δ (CDCl₃): 7.4-7.29, m, 5H, Ph; 6.72, s, 1H, C4-H; 5.56, s, 1H, —CHBr₂; 4.54, bs, 2H, CH₂Ph; 3.0, 1H, C-5 OH, 1.54-0.97, m, C-3 chain. ¹³C n.m.r. δ (CDCl₃): 13.7, 22.3, 29.3, 2.6, 46.8, 91.5, 127.6, 128.3, 128.7, 136.9, 137.0, 160.8, 170.6.

### 1-Benzyl-5-dibromomethyl-3-hexyl-5-hydroxy-1,5-dihydropyrrol-2-one

Method A

A solution of 3-hexyl-5-dibromomethylene-2(5H)-furanone (1.03 g: 3.32 mmol) in benzyl amine (2 ml) was stirred at mom temperature for 0.5 h Dichloromethane (15 ml) was added to the reaction mixture and the precipitated solid was filtered off. The filtrate was washed with aqueous hydrochloric acid (2M, 20 ml), dried over sodium sulfate and evaporated to yield a yellow viscous oil (0.36 g). The crude product was chromatographed on silica using dichloromethane/ethyl acetate (1:19) as the eluent and recrystallised from light petroleum to yield 1-benzyl-5-dibromomethyl-3-hexyl-5-hydroxy-1,5-dihydropyrrol-2-one (0.11 g) as colourless needles m.p. 105-108° C. (Found (HRESMS) m/z 465.994011. C₁₈H₂₃Br₂NO₂Na⁺ (⁷⁹Br) requires 465.998758. νₘₐₓ: 3195, 2987, 2924, 2858, 1676, 1649, 1425, 1153, 1068, 968, 845, 730, 599 cm⁻¹. λₘₐₓ: 205 (εₘₐₓ 7740) nm. ¹H n.m.r. δ(CDCl₃): 7.39-7.26, n, 5H, Ph; 6.7, s, 1H, C4-H, 5.6, s, 1H, —CHBr₂; 4.54, d, J 15 Hz, 2H, CH₂Ph; 2.89-2.35, m, 2H, CH₂, 1.60-0.87, m, 13H, C3-chain. ¹³C n.m.r. δ (CDCl₃): 14, 22.45, 25, 27, 28.8, 31.4, 42,5, 46.7, 91.5, 127.6, 128.5, 128.6, 136.6, 136.7, 144.0, 170.0.

Method B

A mixture of 3-hexyl-5-dibromomethylene-2(5H)-furanone (1.03 g: 3.32 mmol) and benzyl amine (2 ml) in ethanol (5 ml) was stirred at room temperature for 2.5 h. The crude product was isolated and purified as described above to yield 1-benzyl-5-dibromomethyl-3-hexyl-5-hydroxy-1,5-dihydropyrrol-2-one in (72%) yield.

### 1-Butyl-5-dibromomethyl-3-hexyl-5-hydroxy-1,5-dihydropyrrol-2-one

n-Butylamine (0.272 g; 3.72 mmol) was added dropwise to a solution of 5-dibromomethylene-3-hexyl-2(5H)-furanone (0.314 g: 0.93 mmol) in CH₂Cl₂ (10 ml). The mixture was stirred at room temperature for 5 hrs. Column chromatography on silica with CH₂Cl₂ followed by CH₂Cl₂/EtOAc (19:1) afforded the major product as a colourless oil (0.20 g) which upon recrystallisation from petrol gave 1-butyl-5-dibromomethyl-3-hexyl-5-hydroxy-1,5-dihydropyrrol-2-one (52%) as colourless needles, m.p. 85-86°. (Found (HRESMS) m/z 432.013664. C₁₅H₂₅Br₂NO₂Na⁺ (⁷⁹Br) requires 432.014407). νₘₐₓ; 3230, 2957, 2859, 1672, 1650, 1458, 1422, 1375, 1270, 1233, 1139, 1079, 1023, 728, 666, 612 cm⁻¹. λₘₐₓ 259 (⊖ₘₐₓ 945), 206 (9658) nm. ¹H n.m.r. δ(CDCl₃): 6.68, s, 1H, C4-H, 5.8, s, 1H, CHBr₂; 3.45, m, 1H, N—CH₂; 3.10, m, 1H, N—CH₂; 3.15, bs, OH; 3.20, m, 2H, —CH₂; 2.33-2.31, m, —CH₂-chain; 1.65-0.88, m, 14H, alkyl chain. ¹³C n.m.r. δ (CDCl₃): 13.6, 20, 22, 25, 27, 29, 30.75, 31, 39, 46.6, 91.4, 136, 144.5, 170.

### N-(2-Hydroxyethyl)₃-butyl-5-dibromomethyl-5-hydroxy-2(5H)pyrolinone

A solution of ethanolamine (1.13 g; 18.5 mmol) in CH₂Cl₂ (5 ml) was added dropwise to an ice-cooled solution of 3-butyl-5-dibromomethylen-2(5H)-furanone (1.0 g; 9.25 mmol) in dichloromethane. The mixture was stirred at this temperature for 1 hr and then at room temperature for further 1 h. The mixture was washed with waster (3×50 ml), dried over sodium sulfate and evaporated to yield a viscous oil (0.63 g). The cuude product was chromatographed on silica using EtOAc as an eluent to yield N-(2-hydroxyethyl)-3-butyl-5-dibromomethyl-5-hydroxy-2(5H)pyrolinone as an oil which solidified on keeping. Crystallisation from (CH₂Cl₂/petrol) afforded the title compound as colurless needles, m.p. 68-70°. νₘₐₓ: 3439, 3105, 3065, 2957, 2927, 1701, 1593, 1496, 1465, 1370, 1189, 1139, 1095, 1069, 1037, 945, 835, 763 cm⁻¹. λₘₐₓ 203 nm ¹H nmr δ(CDCl₃): 0.93, t, 3H, CH₃; 1.25-1.45, m, 4H, CH₂; 2.35, m, 2H, CH₂; 3.10, m, 1H, NCH₂CH₂OH; 3.84, m, 2H, NCH₂CH₂OH; 4.10, m, 1H, NCH₂CH₂OH; 5.43, bs, 1H, OH, 5.84, s, 1H, CHBr₂; 6.78, s, 1H, H4. ¹³C n.m.r. δ (CDCl₃): 13.73, 22.2, 24.9, 29.3.41.3, 46.7, 61.3, 90.3, 137.5, 142.8, 171.1.

### 5-Dibromomethyl-3-hexyl-5-hydroxy-1,5-dihydropyrrol-2-one

Liquid ammonia (5 ml) was added to 5-dibromomethylene-3-hexyl 2(5H)-furanone (0.50 g; 1.48 mmol) in a sealed tube held in a acetone/liquid nitrogen bath. The reaction mixture was allowed to warm up gradually and kept at room temperature overnight. After gradual evaporation of ammonia the product was extracted with EtOAc (20 ml), washed with water, dried over Na₂SO₄, and evaporated to yield a solid (0.30 g). The crude product was purified on a silica column using first CH₂Cl₂ as the eluent followed by EtOAc/MeOH (4:1). The yellow band upon solvent removal and crystallisation from petrol afforded a yellow crystalline solid (0.07 g) of 5-dibromomethyl-3-hexyl-5-hydroxy-1,5-dihydropyrrol-2-one, m.p. 106-109° C. ¹H n.m.r. δ (CDCl₃): 6.81; s, 1H, C4-H, 6.26, s, 1H, —NH; 5.68, s, 1H, CHBr₂; 3.2, s, C5-OH, 2.28-2.23, m, —CH₂, chain; 1.55-0.91, m, 11H, chain. ¹³C n.m.r. δ (CDCl₃): 13.9, 22, 25.5, 27, 29, 31.4, 129, 140, 142, 170.5.

### 4-bromo-5-hydroxy-5-hydroxymethyl-1,5-dihydropyrrol-2-one

A suspension of 4-bromo-5-bromomethylene-2(5H)-furanone (1.30 g, 5.16 mmol) in aqueous ammonia solution (20% w/w) was stirred at room temperature for ½ h. During this a complete dissolution of furanone was observed. The solution was evaporated to dryness in vacuo at ca 35-40° C., and finally under high vacuum at room temperature. The resulting solid (1.70 g) was recrystallised from ethanol to yield 4-bromo-5-hydroxy-5-hydroxymethyl 1,5-dihydropyrrol-2-one as colourless granules (1.0 g). m.p. 140-142° C. (decomp); νₘₐₓ: 3259, 3100, 2949, 1667, 1592, 1419, 1370, 1152, 1076, 981, 872, 563 cm⁻¹. λₘₐₓ, 220 (εₘₐₓ 6077). ¹H n.m.r. δ (CDCl₃): 8.09, s, —NH; 6.22, d, 2 Hz, H3; 4.97, t, 2 Hz, —CH₂OH; 3.37, q, J 2 Hz, OH; 2.48, d, 2 Hz, CH₂OH. ¹³C n.m.r. δ (CDCl₃): 69.6, 84.3, 132.8, 152.3, 174.1.

### 4-Bromo-3-hexyl-5-hydroxy-5-hydroxymethyl-1,5-dihydropyrrol-2-one

A suspension of 4-bromo-hexyl-5-bromomethylene-2(5H)-furanone (0.50 g; 1.48 mmol) in aqueous ammonia solution (30 mls; 28%) was stirred at room temperature for 2 h, during which time the solid completely dissolved. The solution was evaporated to dryness, and the residue extracted with dichloromethane (25 ml). The organic phase was dried over anhydrous sodium sulfate and evaporated to yield a red viscous oil. Chromatography on silica using ethyl acetate followed by ethyl acetate/methanol (4:1) gave a solid which upon recrystallisation from light petroleum yielded 4-bromo-3-hexyl-5-hydroxy-5-hydroxymethyl-1,5-dihydropyrrol-2-one as colourless granules (0.16 g; 36%), m.p. 134-135°. νₘₐₓ: 3304, 3256, 3185, 2961, 1670, 1589, 1441, 1350, 1136, 1069, 983 cm⁻¹; λₘₐₓ: 221 (εₘₐₓ 6,678), 196 (3,415) nm.

### 5-Ethyl-5-hydroxy-4-methyl-1,5 dihydropyrrol-2-one

A mixture of 5-ethylidene-4-methyl-2(5H)-furanone (0.02 g; 0.162 mmol) in aqueous ammonia solution (5 ml; 28% w/w) was stirred at room temperature for 1.5 h during which time all of the furanone dissolved. The solution was evaporated in vacuo to dryness leaving 5-ethyl-5-hydroxy-4-methyl-1,5-dihydropyrrol-2-one as a white solid (0.015 g; 65%), m.p. 182-186° C. (Found (HRESMS) m/z 164.067448. C₆H₁₁NO₂Na⁺ requires 160.06815). νₘₐₓ: 3204, 2980, 1698, 1664, 1633.5, 1445, 1167, 1080, 1016, 983, 852, 769, 578. λₘₐₓ, 207 (εₘₐₓ 23, 180) nm. ¹H n.m.r. δ (DMSO)-d₆) 7.97, s, 1H, —NH; 5.55, s, 1H, C3-H, 3.18, s, 1H, C5-OH, 1.79, s, 3H, C4-Me; 1.69-1.52, m, 2H, C5-CH₂-Me; 0.34, t, 3H, Me. ¹³C n.m.r. δ (DMSO-d₆): 7.9, 11.9, 29.2, 90.2, 121.7, 162, 171.6.

### 1-Benzyl-5-ethyl-5-hydroxy-4-methyl-1,5-dihydropyrrol-2-one

A solution of 5-ethylidene-4-methyl-2(5H)-furanone (0.124 g; 10 mmol) In benzylamine (0.128 g; 12 mmol) was the left to stand at room temperature for 72 hrs, during which time a solid precipitated from the reaction. The reaction mixture was triturated with CH₂Cl₂/petrol (1:3) and the precipitated solid was filtered and recrystallised from EtOAc/petrol to yield-1-benzyl-5-ethyl-5-hydroxy 4-methyl-1,5-dihydropyrrol-2-one as colourless crystals m.p. 129-132° (70%). νₘₐₓ: 3247, 3082, 2964, 1669, 1638, 1496, 1353, 1101, 1053, 902, 708 cm⁻¹. λₘₐₓ: 276 (εₘₐₓ 2,101), 237 (16,321), 243 (39,646) nm. ¹H n.m.r. δ (CDCl₃): 7.4-7.24, m, 5H, Ph; 5.79, s, 1H, C3-H, 4.46, 2 d, J 15 Hz, —CH₂Ph; 3.81, bs, C5-OH; 1.92, s, C4-Me; 1.83-168, m, C5-CH₂Me; 0.34, t, J 7.51 Hz, C5-CH₂Me. ¹³C n.m.r. δ (CDCl₃): 6.8, 11.85, 26, 41.9, 94, 122, 127, 128, 128.5, 138, 159, 170.

### 5-Aminomethyl-4-heptyl-5-hydroxy-1,5-dihydropyrrol-2-one

5-Bromo-6-bromomethyl-4-heptyl-2(5H)-furanone (0.50 g; 1.47 mmol) was dissolved in liquid ammonia in a sealed tube, and left to stand at room temperature for 72 h. Ammonia was allowed to gradually evaporated leaving behind a yellow crystalline solid. The solid was dissolved in hot ethylacetate (ca 25) ml to remove ammonium bromide and the clear filtrate was concentrated to a small volume (ca 7 ml), to yield 5-minomethyl-4-heptyl-5-hydroxy-1,5-dihydropyrrol-2-one as a crystalline solid, (0.1 g; 34%); m.p. 176° C. νₘₐₓ: 3370, 3248, 2966, 2926, 2855, 1674, 1627, 1469, 1350, 1227, 1095, 1082, 954, 855 cm⁻¹. λₘₐₓ: 208 (εₘₐₓ 6845), 291 (2754) nm. ¹H n.m.r. δ (CDCl₃): 7.53, s, —NH; 5.49, d, C5-CH₂NH₂; 3.35, 3H, m, —C5-OH and —CH₂NH₂; 2.23-2.0, m, CH₂; 1.52-0.85, m, 13H, alkyl chain. ¹³C δ (CDCl₃): 14, 22.4, 26, 26.5, 28.9, 29, 31.6, 66, 73, 78, 120.5, 167.5, 171.6.

### 5-Bromomethyl-4-heptyl-5-hydroxy-1 phenyl 1,5-dihydropyrrol-2-one

5-Bromo-5-bromomethyl-4-heptyl-2(6H)-furanone (0.519; 1.5 mmol) was dissolved in dry aniline (5 ml). The mixture soon solidified; it was allowed to stand at room temperature for 24 h. Dichloromethane (25 mls) was added to the mixture and the organic phase was washed with aqueous hydrochloric acid (2M) and brine. The dried (Na₂SO₄) organic phase was evaporated to yield a yellow solid (0.50 g; 91%). Recrystallisation from light petroleum gave 5-bromomethyl-4-heptyl-5-hydroxy-1-phenyl-1,5-dihydropyrrol-2-one as colourless needles, m.p. 152-154° C. νₘₐₓ: 3194, 2956, 1930, 2854, 1676, 1626, 1589, 1502, 1494, 1393, 1246, 1141, 838, 758, 692 cm⁻¹. λₘₐₓ: 257(εₘₐₓ 3947), 202 (27,313) nm. ¹H n.m.r. δ (CDCl₃): 7.55-7.26, m, 5H, Ph; 5.79, s, C3-H, 4.52, 1H, C5-OH; 3.39, d, 2H, C5-CH₂Br; 2.27-2.12, m, 2H, chain; 1.6-0.91, m, 13H, chain. ¹³C n.m.r. δ (CDCl₃): 14, 22.5, 25.6, 25.8, 29, 29.2, 30.4, 31.6, 121.6, 126, 126.7, 130, 134.6, 163, 170.5.

### 1-Benzyl-5-bromomethyl-4-heptyl-5-hydroxy-1,5-dihydropyrrol-2-one

A mixture of 5-bromo-5-bromomethyl-4-heptyl-2(5H)-furanone (0.51 g, 1.5 mmol) in benzylamine (0.30 g; 2.82 mmol) in ethanol (6 ml) was stirred at room temperature for 1 h. Dichloromethane (25 ml) was added to the reaction mixture and the organic phase was washed with aqueous hydrochloric acid (2M) followed by brine. After drying over sodium sulfate, the solvent was evaporated in vacuo to yield 1-benzyl-5-bromomethyl-4-heptyl-5-hydroxy-1,5-dihydropyrrol-2-one as a viscous oil (0.52 g; 97%) which solidified on standing in the fridge. Colourless needles from light petroleum; m.p. 94-96°. νₘₐₓ: 3270, 3062, 3033, 2957, 2854, 1667, 1637, 1607, 1496, 1416, 1335, 1297, 1257, 1190, 1161, 1140, 1109, 1030, 950, 884, 865, 769 cm⁻¹. λₘₐₓ 251 (εₘₐₓ 2391), 206 (18,974) nm. ¹H n.m.r. δ(CDCl₃): 7.36-7.28, m, 5H. Ph; 5.85, s, C3-H, 4.64 and 3.42, 2d, 2H each, C5-CH₂Br and CH₂Ph; 3.42, bs, 1H, C5-OH, 2.31-2.15, m, 2H, CH₂; 1.62-0.88, m, 13H, alkyl chain ¹³C, n.m.r. δ(CDCl₃): 14, 22.5, 25.5, 26, 29, 29.2, 30.87, 41.9, 122, 127, 128.3, 137.5, 163, 171.

### Synthesis of 3-alkyl-5-halomethylene-1,5-dihydropyrrol-2-one

### 3-Butyl-5-dibromomethylene-1-phenyl-1,6-dihydropyrrol-2-one

Phosphorus pentoxide was added to a solution of 3-butyl-5-dibromomethyl-5-hydroxy-1-phenyl-1,5-dihydropyrrol-2-one in chloroform. The resulting mixture was stirred overnight at room temperature and passed through a pad of Celite. The crude product was chromatographed on silica and recrystallised from light petroleum to yield 3-butyl-5-dibromomethylene-1-phenyl-1,5-dihydropyrrol-2-one as orange needles (78%), orange crystals from petrol. (Found (HRESMS) m/z 419.954622. C₁₆H₁₇Br₂NONa+ (⁷⁹Br) requires 419.955896). λₘₐₓ, 202 (εₘₐₓ 8137), 195 (3850) nm. ¹H n.m.r. δ(CDCl₃): 7.22-7.17, m, 5H, Ph; 7.17, s, C4-H; 2.38-2.36, m, 2H, CH₂; 1.65-0.96, m, C3-chain. ¹³C n.m.r. δ (CDCl₃): 13.6, 22.3, 25.2, 29,5, 128.3, 128.8, 132.1, 139, 140, 171.8.

### 3-Hexyl-5-dibromomethylene-1-phenyl-1,5-dihydropyrrol-2-one

3-Hexyl-5-dibromomethylene-1-phenyl-1,5-dihydropyrrol-2-one was prepared from 3-hexyl-5-dibromomethyl-5-hydroxy-1-phenyl-1,5-dihydropyrrol-2-one as described above. Yellow granules from petrol. νₘₐₓ: 3378, 2957, 2925, 2854, 1692, 1598, 1501, 1492, 1445, 1122, 1081, 743, 677 cm−1. λₘₐₓ: 309 (εₘₐₓ 19,681) nm. ¹H n.m.r. δ (CDCl₃): 7.47.17, m, 6H, Ph and H4; 2.37-2.34, m, 2H, CH₂; 1.57-0.89, m, 11H. C3-chain.

### 1-Benzyl-3-butyl-5-dibromomethylene-1,5-dihydropyrrol-2-one

1-Benzyl-3-butyl-5-dibromomethyl-5-hydroxy-1,5-dihydropyrrol-2-one was dehydrated with P₂O₅ in CHCl₃ at mom temperature for 72 hrs. The mixture was filtered through celite and the solvent evaporated in vacuo to yield a viscous oil, which solidified on keeping in a refrigerator. The solid was recrystallised from methanol/water to yield 1-benzyl-3-butyl-5-dibromomethylene-1,5-dihydropyrrol-2-one as colourless plates, m.p. 56-58° C. (91%). νₘₐₓ: 2954, 1706, 1626, 1495, 1453, 1494, 1435, 1386, 1352, 1269, 1235, 1095, 765 cm−1. λₘₐₓ: 324 (εₘₐₓ: 5985), 283 (16,201), 206 (10,972) nm. ¹H n.m.r. δ(CDCl₃): 7.3-7.07, m, 6H, Ph and H4; 5.26, s, CH₂Ph, 2.4-2.36, m, 2H, CH₂; 1.6-0.95, m, C3-chain. ¹³C n.m.r. δ (CDCl₃): 13.7, 22, 25, 29.6, 44.2, 74.7, 89.25, 126, 127, 128, 132, 137.8, 138.8, 140, 172.1.

### 1-Benzyl-5-dibromomethylene 3-hexyl-1,5-dihydropyrrol-2-one

This compound was prepared according to the procedure described for 1-benzyl-3-butyl-5-dibromomethylene-1,5-dihydropyrrol-2-one. νₘₐₓ; 2960, 2848, 2923, 2854, 1696, 1592, 1496, 1453, 1354, 1316, 977, 830, 738, 630 cm⁻¹. ¹H n.m.r. δCDCl₃): 7.3-7.08, m, 5H, Ph; 7.26, s, 1H, H4; 5.26, 2H, —CH₂Ph; 2.4-2.36, m, 2H, CH₂; 1.56-1.32, m, C3-chain.

### 1-Butyl-5-dibromomethylene-3-hexyl-1,5-dihydropyrrol-2-one

This compound was prepared according to the procedure described for 1-benzyl-3-butyl-5-dibromomethylene-1,5-dihydropyrrol-2-one. Yield (30%). νₘₐₓ; 2956, 2928, 2858, 1705, 1586, 1452, 1360, 1335, 1194, 1135, 1058, 846, 829 741 cm⁻¹; λₘₐₓ: 290 (εₘₐₓ 18,927), 203 (8,409) nm. ¹H n.m.r. δ(CDCl₃): 7.0, s, 1H, C4-H; 3.99-3.93, t, 2H, —CH₂N—; 2.3, t, —CH₂— chain; 1.56-0.88, m, 16H, chain. ¹³C n.m.r. δ (CDCl₃), 13.7, 14, 19.7, 22.4, 25, 27, 29, 31.4, 32.1, 40.6, 132, 137, 139, 140.6, 172.0.

### 5-Dibromomethylene-3-hexyl-1,5-dihydropyrrol-2-one

This product was prepared by the dehydration of 5-dibromomethyl-3-hexyl-5-hydroxy-1,5-dihydropyrrol-2-one as described above, m.p. 103-105°.

### 5-Ethylidene-4-methyl-1,5-dihydropyrrol-2-one

5-Ethyl-5-hydroxy-4-methyl-2(5H)pyrrolinone was dehydrated to 5-ethylidene-4-methyl-1,5-dihydropyrrol-2-one with P₂O₅ in dichloromethane. νₘₐₓ: 3158, 3093, 3036, 1670, 1495, 1434, 1397, 1381, 1356, 1279, 956, 867, 796, 639. λₘₐₓ: 173 (εₘₐₓ 33,010) nm. ¹H n.m.r. δ(CDCl₃): 8.94, s, 1H, —NH; 5.85, 1H, s, C3-H; 5.33, q, J 7.53 Hz, -═CHCH₃; 2.1, s, 3H, C4-Me: 1.92, d, J 7.53, C5-Me-CH═. ¹³C n.m.r. δ (CDCl₃): 11.7, 12.9, 107, 120.5, 140, 148, 172.0.

### 1-Benzyl-5-ethylidene-4-methyl-1,5-dihydropyrrol-2-one

1-Benzyl-5-ethylidene-4-methyl-1,5-dihydropyrrol-2-one was prepared by the dehydration of 1-benzyl-5-ethyl-5-hydroxy-4-methyl-1,5-dihydropyrrol-2-one as described before. λₘₐₓ: 206 (εₘₐₓ2132) nm.

### 5-Bromomethylene-4-heptyl-1-phenyl-1,5-dihydropyrrol-2-one

p-Toluenesulfonic acid (0.05 g) was added to a solution of 5-bromomethyl-5-hydroxy-4-heptyl-1-phenyl-1,5-dihydropyrrol-2-one in toluene. The mixture was refluxed for ½ h and after cooling, washed with sat. NaHCO₃. The organic phase was dried over Na₂SO₄, and evaporated to yield an E,Z mixture of 5-bromomethylene-4-heptyl-1-phenyl-1,5-dihydropyrrol-2-one as a colourless oil which solidified on standing, m.p. 63-65°. νₘₐₓ: 3414, 3080, 2952, 2853, 1695, 1627, 1597, 1499, 1446, 1382, 1269, 1074, 907, 831 cm⁻¹; λₘₐₓ 317 (εₘₐₓ 22,834), 278 (43,910), 204 (46,925) nm; ¹H n.m.r. δ(CDCl₃): 7.4-7.24, m, 5H, Ph, 6.04 and 5.94, 2 s, 1H each, ═CHBr and C3-H, 2.45, m, 2H, CH₂; 1.65-0.9, m, 13H, alkyl chain.

### 1-Benzyl-5-bromomethylene-4-heptyl-1,5-dihydropyrrol-2-one

1-Benzyl-5-bromomethyl-4-heptyl-5-hydroxy-1,5-dihydropyrrol-2-one dehydrated smoothly to an E and Z mixture of 1-benzyl-5-bromomethylene heptyl-1,5-dihydropyrrol-2-one upon heating a solution of 1-benzyl-5-bromomethyl-4-heptyl-5-hydroxy-1,5 dihydropyrrol-2-one with p-toluenesulfonic acid in toluene; m.p. 52-55°; νₘₐₓ: 3096, 2927, 2857, 1704, 1630, 1387, 1357, 954, 855, 843 cm⁻¹; λₘₐₓ: 319 (εₘₐₓ 10,220), 276 (19,433), 206 (17,040) nm; ¹H n.m.r. δ (CDCl₃): 7.29-7.15, m, 5H, Ph; 6.15 and 5.98, 2s, each 1H, ═CHBr and C3-H, 2.39, m, 2H, CH₂; 1.7-0.89, m, 13H, alkyl chain.

### Reaction of N-(2-Hydroxyethyl)-3-butyl-2(5H)pyrolinone with acetic anhydride and Triethylamine

### N-(2-Acetoxyethyl)-3-butyl-5-(dibromomethylene)-2(5H)pyrolinone

A mixture of N-(2-hydroxyethyl)-3-butyl-5-dibromomethyl-5-hydroxy-2(5H)pyrrolinone (0.2 g, 0.54 mmol), acetic anhdride (0.44 g; 4.4 mmol) and triethylamine (0.44 g; 4.4 mmol) in dry dichloromethane (10 ml) was refluxed for 2 hr. After cooling to room temperature, the mixture was washed with aqueous sodium bicarbonate and brine. The organic phase was dried over anhydrous sodium sulfate and evaporated to yield a viscous oil. ¹H n.m.r showed it to be a mixture of the mono- (88%) and di-acetate (12%) derivatives. Chromatography on silica using EtOAc/CH₂Cl₂ (5:1) as an eluent yielded 5-acetoxy-N-(2-acetoxyethyl)-3-butyl-5-dibromomethyl-2(5H)pyrrolinone (12%) as an oil. νₘₐₓ: 2957, 2931, 2875, 1766, 1720, 1433, 1369, 1236, 1044, 1013, 855, 707 cm¹. λₘₐₓ 217 (εₘₐₓ 1692), 268 (738) nm. ¹H n.m.r. δ (CDCl₃) 0.91 (t, 3H, CH₃); 1.38 (m, 2H, CH₂); 1.55 (m, 2H, CH₂); 2.05 and 2.10 (each s, 3H, CH₃); 2.34 (m, 2H, CH₂); 3.61 (m, 1H, NCH₂CH₂); 3.64 (m, 1H, NCH₂CH₂); 4.27 (m, 2H, NCH₂CH₂); 6.26 (s, 1H, CHBr₂); 6.83 (s 1H, H4). ¹³C n.m.r. δ(CDCl₃) 13.7, 20.8, 21.2, 22.1, 24.9, 38.7, 44.1, 61.5, 94.1, 134.2, 144.3, 168.4, 170.6, 171.0. N-(2-acetoxyethyl)-3-butyl-5-dibromomethyl-5-hydroxy-2(5H)pyrrolinone (88%) 1H n.m.r. δ (CDCl₃) 0.93 (t, 3H, CH₃); 1.38 (m, 2H, CH₂); 1.55 (m, 2H, CH₂): 2.21 (s, 3H. CH₃); 2.34 (m, 2H, CH₂); 3.27 (m, 1H, NCH₂CH₂); 4.04 (m, 2H, NCH₂CH₂); 4.30 (s, 1H. OH); 4.62 (m, 1H, NCH₂CH₂); 5.85 (s, 1H, CHBr₂); 6.73 (s, 1H, H4). ¹³C n.m.r. δ(CDCl₃) 13.7, 20.9, 22.2, 24.9, 29.3, 38.1, 45.9, 62.5, 91.0, 137,4, 143.3, 170.4, 171.9.

Dehydration of N-(2-acetoxyethyl)-3-butyl-5-dibromomethyl-5-hydroxy-2(5H)pyrrolinone with p-toluenesulfonic acid in toluene gave quantitatively N-(2-acetoxyethyl)₃-butyl-5-(dibromomethylene)₂(5H)pyrolinone. νₘₐₓ, 2957, 2929, 2870, 1744, 1705, 1441, 1368, 1229, 1177, 1161, 1130, 1035, 830, 764 cm⁻¹. ¹H n.m.r. δ(CDCl₃) 0.93 (t, 3H, CH₃): 1.36 (m, 2H, CH₂); 1.55 (m, 2H, CH₂); 2.02 (s, 3H, CH₃); 2.32 (m, 2H, CH₂); 4.25-4.31 (m, 4H, NCH₂CH₂); 7.05 (s, 1H, H4). ^{—}C n.m.r. δ(CDCl₃) 13.7, 20.7, 22.3, 25.1, 29.5, 39.5, 62.3, 73.8, 132.4, 138.6, 140.3, 170.6, 172.0.

### Hydrolysis of N-(2-acetoxyethyl)-3-butyl-2(5H)pyrolinone N-(2-hydroxyethyl)-3-butyl-5(dibromomethylene)-2(5H)pyrolinone

A solution of potassium carbonate (1 g) in water (3 ml) was added dropwise to a solution of N-(2-acetoxyethyl)₃-butyl-5-(dibromomethylene)₂(5H)pyrolinone (0.2 g, 0.51 mmol) in methanol (7 ml). After stirring the mixture at room temperature for 20 mins, methanol was removed in vacuo and the product extracted with ethylacetate (2×40 ml). The resulting extracts were combined, washed with brine, dried (Na₂SO₄), and evaporated to yield an oil (0.18 g; 94.5%), which solidified upon standing in the fridge. Crystallisation from light petroleum gave N-(2-hydroxyethyl)-3-butyl-5-dibromomethylene-2(5H)pyrolinone as colurless granules, m.p. 48-50° max 3404, 2957, 2930, 2880, 1720, 1651, 1465, 1348, 1207, 1081, 1054, 1018, 936, 850, 716 cm⁻¹. νₘₐₓ 206 (εₘₐₓ 25,389), 239 (6,758), 288 (2,186) nm. ¹H n.m.r. δ(CDCl₃) 0.91 (t, 3H, CH₃); 1.36 (m, 2H, CH₂); 1.54 (m, 2H, CH₂); 2.29 (m, 2H, CH₂); 3.83 (m, 2H, NCH₂CH₂); 4.20 (m, 2H, NCH₂CH₂); 7.02 (s, 1H, H4). ¹³C n.m.r. δ (CDCl₃) 13.7, 22.3, 24.9, 29.5, 43.3, 46.8, 61.9, 74.5, 132.3, 138.6, 140.5, 173.2.

### Synthesis of 5-phenylaminomethylene-2(5H)furanone

### 4-Bromo-5-phenylaminomethylene-2(5H)-furanone

A solution of 4-bromo-5-bromomethylene-2(5H)-furanone (0.30 g; 0,79 mmol) was dissolved in aniline (6 ml), and left to stand at room temperature for 3 hrs, during which time the mixture solidified. The solid was triturated with CH₂Cl₂/petrol (1:1; v/v, 20 ml) and filtered. The resulting solid was dried and recrystallised from ethanol to yield 4-bromo-5-phenylaminomethylene-2(5H)-furanone (0.24 g, 49%) as yellow needles, m.p. 200-202° C. (decomp). (Found (HRESMS) m/z 287.963053. C₁₁H₈BrNO₂Na⁺ (⁷⁹Br) requires m/z 287.963840). νₘₐₓ 3233, 3127, 1730, 1697, 1595, 1498, 1276, 1195, 932, 798, 756 cm⁻¹. λₘₐₓ 397 nm (εₘₐₓ 50,686); 246 (12,769), 202 (15,961). ¹H n.m.r. δ (CDCl₃): 9.99, d, J 10.44 Hz, 1H, NHPh; 7.31-6.99, m, Ph; 7.07, d, J 10.44 Hz, 1H, ═CHNHPh; 6.16, s, C3-H. ¹³C n.m.r. δ (CDCl₃): 109.0, 116.2, 117.9, 129.9, 129.8, 133.9, 167.5.

### 5-Phenylaminomethylene-4-bromo-3-butyl-2(5H)-furanone

A solution of 4-bromo-3-butyl-5-bromomethylene-2(5H)-furanone (0.25 g; 0.81 mmol) in aniline (0.082 g; 0.88 mmol) was left to stand at room temperature for 72 h. The mixture was diluted with CH₂Cl₂ (50 ml), washed with aqueous hydrochloric acid (2M) and dried over anhydrous sodium sulfate. The solvent was removed in vacuo leaving behind a brown viscous oil (0.29 g). The crude product was chromatographed on silica using dichloromethane to yield 5-phenylaminomethylene-4-bromo-3-butyl-2(5H)-furanone as a yellow solid. ¹H n.m.r δ(CDCl₃): 7.40-6.80, m, 5H, Ph; 6.70, d J 12.5 Hz, ═CH(NH)Ph; 2.42-2.40, m, 2H, CH₂-chain; 17-1.2, m, 4H, CH₂-chain; 0.95, t J 7.3 Hz, CH3. (Found (HRESMS) m/z 344.021931. C₁₅H₁₆BrNO₂Na⁺ (⁷⁹Br) requires m/z 344.021891).

### 4-Bromo-5-phenylaminomethylene-3-hexyl-2(5H)-furanone

A mixture of 4-bromo-3-hexyl-5-bromomethylene-2(5H)-furanone (0.503; 1.48 mmol) and aniline (1 ml) in ethanol (10 ml) was heated at reflux for 2 h. After cooling to room temperature, the mixture was evaporated to dryness and the residue extracted with dichloromethane (20 ml). The organic phase was washed with aqueous hydrochloric acid (2M) and dried over anhydrous sodium sulfate. Removal of the solvent and recrystallisation of the solid form light petroleum gave 3-bromo-5-phenylaminomethylene-3-hexyl-2(5H)-furanone (0.50 g: 100%) as yellow needles; m.p. 147-148° C., νₘₐₓ 3242, 3161, 3109, 29212, 2842, 1728, 1683, 1600, 1581, 1500, 1350, 1236, 1055, 960, 750, 673 cm⁻¹. λmax: 394 (εₘₐₓ 26,287), 247 (8002) nm. ¹H n.m.r. δ(CDCl₃): 7.32-6.97, m, 5H, Ph: 6.98, s, —NHPh; 6.73, s, C5 ═CH—NHPh; 2.4, t, —CH₂-chain; 1.61 0.88, m, 11H, chain. ¹³C n.m.r. δ(CDCl₃): 14.0, 22.0, 24.8, 27.6, 29, 31.0, 103.0, 113.0, 115.0, 122.5, 124.0, 129.5, 129.5, 131.0, 139.9, 167.0.

### 5-Phenylaminomethylene-4-heptyl-2(5H)furanone

### 5-Phenylaminomethyl-4-heptyl-5-hydroxy-2(5H)pyrrolinone

5-Bromomethylene 4-heptyl-2(5H)-furanone (0.44 g; 1.61 mmol) was dissolved In dry aniline (2 ml) and left to stand at room temperature for 24 h. Dichloromethane (10 ml) was added to the reaction mixture and the organic phase was washed with aqueous hydrochloric (2M) followed by water. After drying over sodium sulfate, the solvent was evaporated off to yield a pale yellow solid. The crude product was chromatographed on silica column using dichloromethane followed by CH₂Cl₂/EtOAc (2:1; v:v) as the eluents to yield 5-phenylaminomethyl-4-heptyl-5-hydroxy-2(5H)-furanone (0.43 g; 88%) as a pale yellow solid, m.p. 172-174° C. νₘₐₓ: 3192, 3037, 2957, 2931, 2953, 1676, 1643, 1598, 1502, 1493, 1336, 1250, 1160, 923, 757 cm⁻¹. λₘₐₓ 278 (εₘₐₓ 7188), 203 (8609) nm. ¹H n.m.r, δ (CDCl₃): 7.53-7.26, 6H, Ph and —NHPh; 5.73, s, 1H, C3-H, 5.11, s, 1H, C5-OH; 3.37, d, 2H, —CH₂NHPh; 2.2-2.0, m, 2H, —CH₂-chain; 1.25-0.91, m, 13H, chain. ¹³C n.m.r. δ(CDCl₃): 14.0, 22.6, 25.5, 25.3, 25.8, 29.0, 29.2, 30.4, 31.6, 93.4, 121.8, 126.0, 126.7, 129.0, 134.6, 163.0, 170.4.

### 5-Phenylaminomethylene-4-heptyl-2(5H)-furanone

A sample of 5-phenylaminomethyl-4-heptyl-5-hydroxy-2(5H)-furanone was dehydrated using p-toluenesulfonic acid in toluene to yield an E and Z mixture of 5-phenylaminomethylene-4-heptyl-2(5H)-furanone as a colourless oil which solidified on standing in the fridge. νₘₐₓ: 3088(—NH), 3052, 2927, 2866, 1712, 1626, 1598, 1499, 1464, 1264, 1195, 759, 699 cm⁻¹. λₘₐₓ 292 (εₘₐₓ 7623), 204 (4728) nm. ¹H n.m.r. δ(CDCl₃): 7.4-7.25, 6H, Ph and NHPh; 6.19-6.1, d, 1H, C3-H, 5.93-6.0, 1H, d, C5- ═CHNHPh; 1.68-0.90, 15H, chain. ¹³C n.m.r. δ(CDCl₃): 14, 22.5, 26.4, 28.1, 28.9, 29.0, 29.2, 30.0, 31.6, 31.7, 88.7, 93.0, 118.5, 122.6, 127.8, 128.2, 128.4, 128.6, 128.7, 129.3, 129.6, 134.0, 135.0, 142.0, 143.0, 152.0, 153.2, 168.0.

### 4-Methyl-5-(1-phenylamino-ethylidene)-5H-furan-2-one

A solution of 5-ethylidene-methyl-2(5H)-furanone (0.31 g; 2.5 mmol) in aniline (0.26 g; 2.75 mmol) was left to stand at r.t. for 3 hrs, during which time a solid precipitated from the reaction. The reaction mixture was triturated with CH₂Cl₂/petrol (1:1) and the solid filtered and recrystallised from EtOAc/petrol to yield 5-ethyl-5-hydroxy-4-methyl-1-phenyl-1,5-dihydropyrrol-2-one as colourless crystals (70%); m.p. 97-100°. νₘₐₓ: 3287, 1884, 1704, 1530, 1496, 1353, 1101, 1053, 971, 897, 790, 756, 688, 638 cm⁻¹. λₘₐₓ: 273 (εₘₐₓ 15,256), 226 (16,382), 243 (39,646) nm. ¹H n.m.r. δ (DMSO-de) 10.11, s, 1H, —NH; 7.57, d, 2H, ArH; 7.30, t, 3H, ArH; 6.08, s, 1H, CH; 3.27, s, 3H, CH₃; 1.96, s, 3H, CH₃. ¹³C n.m.r. δ (CDCl₃): 20.9, 119.6, 123.7, 123.8, 129.1, 139.3, 142.9, 163.1, 170.4.

### Synthesis of 5-arylamino and arylalkylamino-2(5H)furanones 4-bromo-5-benzylamino-5-bromomethyl-2(5H)-furanone

Benzyl amine (0.10 g; 0.95 mmol) was added with stirring to an ice-cooled solution of the 4-bromo-5-(bromomethylene)-2(5H)-furanone (0.16 g; 0.64 mmol) in dichloromethane (10 ml). The mixture was stirred at room temperature for 2.5 h, washed with aqueous hydrochloric acid solution (1 M, 10 ml), dried (Na₂SO₄), and evaporated to yield a brown oil. The crude product was chromatographed on silica using dichloromethane/ethyl acetate (1:4; v:v) as the eluent and recrystallised from dichloromethane/light petroleum to yield 4-bromo-5-benzylamino-5-bromomethyl-2(5H)-furanone as orange flakes. m.p. 137-139° (Found (HRESMS) m/z 381.901032. C₁₂H₁₁Br₂NO₂Na⁺ (⁷⁹Br) requires 381.904812). νₘₐₓ 3256, 1674, 1655, 1431, 1413, 1352, 1072, 1054, 699 cm⁻¹. λₘₐₓ 257 (εmax 2879) nm. ¹H n.m.r. δ (CDCl₃): 7.38, d, J 11 Hz, 1H, —NHCH₂—; 7.37-7.29, m, Ph; 6.38, s, C3H, 4.65, d, J 15 Hz, 1H, —CH₂Br; 4.44, d, J 15 Hz, 1H, —CH₂Br and 3.58-3.44, dd, J 15 Hz, CH₂Ph. ¹³C n.m.r. δ(CDCl₃): 30.6, 42.8, 53.0, 922, 128.0, 128.2, 128.9, 137.0, 142.0, 168.0.

### 4-bromo-5-benzylamino-5-bromomethyl-3-hexyl-2(5H)-furanone

Benzylamine (0.32 g; 2.96 mmol) was added with stirring to a solution of 4-bromo-3-hexyl-5-bromomethylene-2(5H)-furanone (0.50 g; 1.48 mmol) in ethanol (6 ml). The mixture was stirred at room temperature for 1 h and evaporated to dryness. The residue was extracted with dichloromethane (20 ml) and the dichloromethane extract washed with aqueous hydrochloric acid (2M). After drying over anhydrous sodium sulfate, removal of the solvent gave a thick viscous oil, Column chromatography on silica gel using dichloromethane followed by dichloromethane/ethyl acetate (19:1) as the eluents afforded 4-bromo-5-benzylamino-5-bromomethyl-3-hexyl-2(5H)-furanone (0.36 g; 56%) as a viscous oil; m.p. 72-75°. νₘₐₓ 3277, 3065, 3032, 2954, 2928, 2857, 1681, 1496, 1411, 1355, 1151, 1064, 1104, 1030, 988, 907, 726, 698. λₘₐₓ: 277 (εₘₐₓ: 39,542), 205 (38,034) nm. ¹H n.m.r. S(CDCl₃): 7.47.26, m, Ph; 4.84.74, d, and 4.4, d, C5-CH₂Br; 3.6 and 3.53, d, C5-NHCH₂Ph; 2.42-2.33, m, —CH₂, chain; 1.56-0.85, m, 11H, chain. ¹³C n.m.r. δ(CDCl₃): 22.0, 25.0, 27.0, 28.8, 31.4, 42.9, 46.7, 49.5, 90.6, 91.6, 127.0, 128.0, 129.0, 136.0, 136.7, 136.9, 138.0, 140.0, 144.0, 168.0, 170.6.

### 5-Phenylamino-3,5-dimethyl-2(5H)-furanone

Method A

A solution of 3,5-dimethyl-5-hydroxy-2(5H)-furanone (0.13 g; 1.02 mmol) in dry aniline (2 mls) was stirred at room temperature for 1 hr. A thin layer chromatography analysis of the mixture (developing solvent; CH₂Cl₂) indicated completion of the reaction as indicated by the disappearance of the starting material. Dichloromethane (25 mls) were added to the mixture and the solution washed with aqueous hydrochloric acid solution (1M; 3×20 mls). The organic layer was dried over anhydrous sodium sulfate and evaporated to yield 5-phenylamino-3,5-dimethyl-2(5H)-furanone as a viscous oil which solidified on keeping (0.013 g). A sample was recrystallised from dichloromethane/light petroleum to yield the furanone as colourless needles νₘₐₓ 3360, 3088, 2965, 1770, 1601, 1570, 1536, 1294, 1246, 1132, 1040, 999, 867, 756, 697 cm⁻¹. λₘₐₓ 236 nm. ¹H n.m.r. δ (CDCl₃): 7.18, t, 2H Ph; 6.90, t, 1H, ArH, 6.89, s, 1H, 14, 6.83, d, 2H, ArH; 4.24, bs, 1 h, OH; 1.91, s, 3H, C3-Me; 1.75, s, 3H, -Me. ¹³C n.m.r. δ (CDCl₃): 10.4, 26.2, 95.5, 121.3, 122.7, 128.9, 132.4, 133.5, 141.9, 148.8, 156.5, 171.9.

Method B

A mixture of 3,5-dimethyl-5-hydroxy-2(5H)-furanone (0.13 g; 1.02 mmol) and aniline (2 ml) in dry toluene (10 ml) was refluxed for 5 h. The mixture was cooled and evaporated. The residue was dissolved in dichloromethane (25 ml) and the solution washed with aqueous hydrochloric acid solution (1M; 3×20 ml). The organic layer was dried over anhydrous sodium sulfate and evaporated to yield 5-phenylamino-3,5-dimethyl-2(5H)-furanone as a viscous ml). The crude product was chromatographed on silica using dichloromethane/ethyl acetate (19:1) as the eluent (Yield 58.0%).

### 5-Phenylamino-5-methyl-4-phenyl-2(5H)-furanone

A mixture of 5-hydroxy-5-methyl-4-phenyl-2(5H)-furanone (0.13 g; 1.02 mmol) and aniline (2 ml) in dry toluene (10 ml) was refluxed for 5 h. The mixture was cooled and washed with aqueous hydrochloric acid solution (2M; 3×20 mls). The organic layer was dried over anhydrous sodium sulfate arid evaporated to yield a viscous oil. The crude product was chromatographed on silica using dichloromethane/ethyl acetate (19:1) as the eluent and recrystallised from dichloromethane/light petroleum to yield 5-phenylamino-5-methyl-4-phenyl-2(5H)-furanone (0.10 g; 72%) as colourless flakes. m.p. 158-160° C. νₘₐₓ: 3356, 1724, 1608, 1534, 1501, 1320, 1291, 1376, 1030, 943, 846, 770, 756, 691, 639. λₘₐₓ, 276(⊖ₘₐₓ 7056), 238 (5615) nm. ¹H n.m.r. δ (CDCl₃): 7.94-7.44, m, 5H, -Ph; 7.14-6.82, m, 5H, Ph; 6.4, s, 1H, C3-H, 4.53, bs, 1H, —NHPh; 1.9, s, C5-Me. ¹³C n.m.r δ (CDCl₃): 117.3, 120, 122.5, 128, 129.5, 131, 142, 159, 166, 170.

### 5-Benzylaminomethyl-3-methyl-2(5H)-furanone

Phosphorus pentoxide (2 g) was added to a solution of 3,5-dimethyl-5-hydroxy-2(5H)-furanone (0.50 g; 2.15 mmol) in dichloromethane (25 ml). The mixture was refluxed for 2 h and the cooled solution was filtered through celite and evaporated in vacuo to yield 3-methyl-5-methylene-2(5H)-furanone as a colourless oil (0.37 g; 82%). The methylene product was dissolved in dichloromethane (5 ml) and benzylamine (1.15 g; 10.8 mmol) was added at room temperature. The mixture was stirred at room temperature for 1 h. After evaporation of the solvent the crude product was chromatographed on silica using dichloromethane/light petroleum as the eluent to yield 5 benzylaminomethyl-3-methyl-2(5H)-furanone as a colourless oil (0.12 g; 26%). νₘₐₓ: 2929, 2854, 1788, 1747, 1715, 1618, 1456, 1388, 1373, 845, 712 cm⁻¹. λₘₐₓ: 308 nm (εₘₐₓ; 1462), 260 (5243). ¹H n.m.r. δ (CDCl₃): 7.29-7.21, m, 6H, Ph and NHCH₂Ph; 6.65, s, 1H, C₄—H, 4.82, s, 2H, —CH₂Ph; 4.70, d, CH₂NHPh; 2.02, s, O₃-Me. ¹³C n.m.r. δ (CDCl₃): 10.8, 25.9, 42.9, 95.0, 105.3, 126.9, 127.1, 128.5, 131.2, 134.2, 137.2, 148.4.

### Side-chain functionalization 3(1′-Bromohexyl)-1)-1-butyl-5-dibromomethylene-1,5-dihydropyrrol-2-one

N-Bromosuccinimide (0.32 g; 1.79 mmol) was added to a solution of 1-butyl-5-dibromomethyl-3-hexyl-1,5-dihydropyrrol-2-one (0.64 g; 1.63 mmol) containing few crystals of benzoyl peroxide in CCl₄ (25 ml). The mixture was heated at reflux under a 100 watt fluorescent lamp for 24 h. The reaction mixture was cooled and passed through a pad of Celite. The filtrate was evaporated to dryness to yield a brown oil which was chromatographed on a silica column using CH₂Cl₂/petrol (1:1) as the eluent to yield 3(1′-Bromohexyl)-1-Butyl-5-dibromomethylene-1,5-dihydropyrrol-2-one (0.46 g; 59.8%) as a pale yellow oil. (Found): HRESMS): m/z 483.849575. C₁₅H₁₄NB₄O₃) requires 483.851758. νₘₐₓ: 3017, 2950, 1709, 1598, 1593, 1480, 1215, 1194, 845, 695, 668 cm⁻¹. λₘₐₓ: 326 (⊖max 4070) nm. ¹H n.m.r. δ(CDCl₃): 7.28, s, 1H, H4; 4.78, t, —CHBr-chain; 2.19-2.11, m, —CH₂-chain; 1.53-0.98, m, alkyl chain. ¹³C n.m.r. δ(CDCl₃): 13.12, 20.95, 26.8, 39, 43.9, 79.5, 9.5, 128.6, 128.9, 129.4, 133.8, 134.5, 138.2, 139.6, 168.7.

### 3-(1′-Bromobutyl)-1-butyl-5-dibromomethylene-1,5-dihydropyrrol-2-one

N-Bromosuccinimide (0.32 g; 1.79 mmol) was added to a solution of N-butyl-5-dibromomethyl-3-hexyl-2(5H)pyrrolinone (0.64 g; 1.63 mmol) containing few crystals of benzoyl peroxide (0.01 g) in CCl₄ (25 ml). The mixture was heated at reflux under a 100 watt fluorescent lamp for 24 h. The reaction mixture was cooled and passed through a pad of Celite. The filtrate was evaporated to dryness to yield a semi-solid (0.69 g) which was chromatographed on a silica column using CH₂Cl₂/petrol (1:1) as the eluent to yield 3-(1′-bromobutyl)-1 butyl-5-dibromomethylene-1,5-dihydropyrrol-2-one (0.46 g; 60%) as a pale yellow oil. λₘₐₓ: 2930, 2957, 2871, 1705, 1684, 1357, 1192, 1055, 902, 769, 651 cm⁻¹; λₘₐₓ: 325 (εₘₐₓ 11,669), 202 (9,879) nm. 1H n.m.r. δ(CDCl₃): 7.29, d, 1H, C4-H, 4.78, t, 1H, C3-CHBr— chain; 3.98, t, 2H, >NCH₂; 2.10, m, —CHOW chain; 1.58-0.93, m, 12H, chain; ¹³C n.m.r. δ(CDCl₃): 13.78, 13.96, 19.8, 22.4, 27.4, 31, 32.2, 37, 41, 43.9, 98.7, 132.5, 138.2, 140, 169.0.

### 3(1′-Bromobutyl)-5-dibromomethylene-N-phenyl-1,5-dihydropyrrol-2-one

N-Bromosuccinimide (0.056 g; 0.316 mmol) was added to a solution of 5-dibromomethyl-3-butyl-1-phenyl-1,5-dihydropyrrol-2-one (0.64 g; 1.63 mmol) containing few crystals of benzoyl peroxide (0.01 g) in CCl₄ (10 ml). The mixture was heated at reflux under a 100 waft fluorescent lamp for 24 h. The reaction mixture was cooled and passed through a pad of Celite. The filtrate was evaporated to dryness to yield a brown oil (0.17 g) which was chromatographed on a silica column using CH₂Cl₂/petrol (1:1) as the eluent to yield 3-(1′-bromobutyl)-5-dibromomethylene-1-phenyl-1,5-dihydropyrrol-2-one as a pale viscous oil (0.10 g). (Found: HRESMS) m/z: 483.849576, C₁₅H₁₄Br₃NONa⁺ (Br⁷⁹) requires 483.851758. νₘₐₓ: 3017, 2950, 1709, 1598, 1593, 1480, 1215, 1194, 1122, 845, 756, 695, 668 cm⁻¹. λₘₐₓ: 326 (εₘₐₓ 3,896), 202 (5,566) nm. 1H n.m.r. δCDCl₃); 7.45, m, 6H, Ph and C3-H, 4.86, t, 1H, C3-CHBr— chain; 2.16, m, —CH₂ chain; 1.53-0.98, m, 5H, alkyl chain. ¹³C n.m.r. δ(CDCl₃): 13, 21, 26.8, 39, 43, 79.5, 95, 107, 128.6, 129.4, 134, 134.5, 138, 139.6, 169.

### N-Phenyl-3-(1′-hydroxybutyl)₅-dibromomethylene-2(5H)pyrrolinone

A solution of N-Phenyl-3-(1-bromobutyl)-5-dibromomethylene-2(5H)pyrolinone (0.0194 mol) in DMSO (60 ml) containing few drops of water was left to stand aside at room temperature for 6 days. The mixture was diluted with dichloromethane (100 ml) and the resulting solution washed with brine (3×120 ml). The organic phase was dried over anhydrous sodium sulfate and evaporated to yield a pale yellow oil (9.08 g). The crude product was purified on a silica column using initially dichloromethane followed by dichloromethane/ethyl acetate to afford N-phenyl-5-dibromomethylene-3(1′-hydroxybutyl)-2(5H)pyrolinone (6.83 g; 88%) as pale yellow needles (dichloromethane/light petroleum), m.p. 93-95°. νₘₐₓ 3439, 3065, 2957, 2927, 2871, 1701, 1496, 1455, 1370, 1189, 1139, 1095, 1069, 1038, 945, 835, 763, 697 cm⁻¹. λₘₐₓ 203 (εₘₐₓ 11,968), 313 (10,707) nm. ¹H n.m.r. δ (CDCl₃) 0-97 (t 3H, CH₃); 1.39 (m, 2H, CH₂); 1.79 (m, 2H, CH₂); 4.61, m, 1H, H1′; 2.71, bs, 1H, OH; 7.23 (s, 1H, H4); 7.21-7.44 (m, 5H, ArH). ¹³C n.m.r. δ(CDCl₃) 13.7, 14, 18.5, 37.8, 67.4, 128.6, 128.9, 128.3, 129.5, 131.5, 134.5, 139.8, 170.8.

### N-Phenyl-3(1′-acetoxybutyl)-5-dibromomethylene-2(5H)pyrrolinone

A solution of acetyl chloride (0.25 ml, 3.2 mmol) in dichloromethane (3 ml) was added dropwise to an ice-cooled solution of N-Phenyl-3-(1-hydroxybutyl-5-dibromomethylene-2(5H)pyrolinone (0.1 g, 0.25 mmol) in dichloromethane (10 ml) containing triethylamine (0.25 ml, 2.47 mmol). The mixture was stirred in ice for 1 h and then at room temperature overnight. The mixture was poured into saturated sodium bicarbonate solution (20 ml) and extracted with dichloromethane (3×30 ml). The organic phase washed with water (3×20 ml), dried over anhydrous sodium sulfate and evaporated to yield a pale yellow oil (0.11 g). The crude product was purified on a silica column using dichloromethane/ethyl acetate (15:1) to afford N-phenyl-5-dibrommethylene-3(1′-acetoxybutyl)-2(5H)pyrolinone (0.1 g) as a viscous oil νₘₐₓ: 2960, 2931, 2873, 1753, 1712, 1592, 1494, 1454, 1362, 1262, 1232, 1193, 1148, 1096, 1060, 836, 753, 698 cm⁻¹. λₘₐₓ 281, 321 nm. ¹H n.m.r. δ (CDCl₃) 0.96 (t, 3H, CH₃); 1.38 (m, 2H, CH₂); 1.91 (m, 2H, CH₂); 5.72, m, 1H, H1′; 7.21 (s, 1H, H4); 7.21-7.40 (m, 5H, ArH).

### Malonic acid mono-[1-(5-dibromomethylene-2-oxo-1-phenyl-2,5-dihydro-1H-pyrrol-3-yl)-butyl]ester

A solution of N-Phenyl-3-hydroxybutyl)-5-dibromomethylene-2(5H)pyrolinone (0-5 g, 1.25 mmol) in dichloromethane (15 ml) containing triethylamine (0.25 ml, 2.47 mmol) was added drop wise over a period of 9 h to an ice-cooled solution of malonyl dichloride (0.36 g, 25 mmol) in dichloromethane (10 ml). The mixture was allowed to stand at room temperature overnight, washed with brine (3×20 ml), dried over anhydrous sodium sulfate and evaporated to yield a brown viscous oil. The crude product was purified on a silica column using ethyl acetate/methanol (1:4) to afford malonic acid mono-[1-(5-dibromomethylene-2-oxo-1-phenyl-2,5-dihydro-1H-pyrrol-3-yl)butyl]ester (0.48 g) as a viscous oil νₘₐₓ 3470, 2959, 2873, 1709, 1594, 1494, 1454, 1362, 1245, 1194, 1148, 1096, 1060, 835, 753, 698 cm⁻¹. λₘₐₓ 271,303 nm. ¹H n.m.r δ(CDCl₃) 0.96 (t, 3H, CH₃); 1.46 (m, 2H, CH₂); 1.88 (m, 2H, CH₂); 3.01, m, 2H, COCH₂CO; 5.67, m, 1H, H1′; 7.23, m, 2H, ArH; 7.43 (m, 3H, ArH); 7.59, s, 1H, H4.

### Preparation of 2(5H)pyrrolinone-polystyrene copolymer

A mixture of styrene (7.13 g), 3(1′-Bromobutyl)-5-dibromomethylene-N-phenyl-1,5-dihydropyrrol-2-one (0.37 g) and AlBN (0.026 g) was degassed for ½ h bby purging with argon gas and then heated at 65° C. for 3 h. After completion of polymerisation, the mixture was poured into methanol and precipitated polymer was filtered, washed extensively with methanol and dried to yield the copolymer (2.38 g, 32%).

### Surface attachment of 2(5H)pyrrolinone

A layer of malonic acid mono-[1-(5-dibromomethylene-2-oxo-1-phenyl-2,5-dihydro-1H-pyrrol-3-yl)-butyl]ester was covalently attached to a surface containing amino groups by immersing the surface in a solution of 2(5H)pyrrolinone (2 mg/ml) in acetonitrile/water containing NHS, N-hydroxy succinimide. The mixture was shaken for 10 minutes and EDC, N-(3-dimethylaminopropyl)-N′-ethylacrbodiimide hydrochloride, was added to the solution to give a final concentration of (2 mg/ml). After shaking the solution for 24 h, the surface was taken out of the solution and washed thoroughly with water and dried. The surface analysis was performed using XPS and % bromine was used as a marker for determining the extent of covalent attachment.

Biological Activity of Furanones

Effect of Furanones as Inhibitor of AHL-Mediated Quorum Sensing, Al-2 pathway and Growth of S. aureus

Methods

Gfp Assay

Briefly, the Gfp assay determines the relative effectiveness of a compound as an inhibitor of AHL mediated quorum sensing. The assay is dependent on a bacterial strain that carries a reporter plasmid. This plasmid expresses the green fluorescent protein (Gfp) in the presence of AHLs (2). The presence of a competitor will prevent AHL mediated Gfp expression of the reporter. The assay can be used to generate an index of inhibition for each compound. The results here, presented as good, moderate, or poor, are based on the index of each of the compounds as an inhibitor of AHL mediated quorum sensing using this bioassay.

Attachment/Biofilm Formation

The ability of furanones to inhibit biofilm formation or attachment has been determined using a modification of the 96 well microtitre method described by Christensen et al. ((1)). The furanones are added to the wells of the microplate and the solvent is allowed to evaporate, leaving the furanones adsorbed onto the plate. Then a suspension of the monitor bacterium, Pseudomonas aeruginosa, is added to each well and incubated for 24 h. Following incubation, the wells are rinsed to remove unattached or loosely adhered cells. The attached wells are fixed with formaldehyde and subsequently stained with cyrstal violet. Following extensive washing to remove the crystal violet, the wells are read at 600 nm. The attachment/biofilm formation in the presence of the furanones is calculated as the percentage of the controls, which are not exposed to the furanones.

Two-Component Signal Transduction Assays

Taz-1 Assay

The Taz-assay carried out according to the method of Jin and Inouye (1993) with the following alterations E. coli RU1012 (pYT0301) were grown overnight In M9 medium at 37° C. supplemented with 100 ug/ml ampicillin and 50 ug/ml kanamycin. This overnight culture was then used to inoculate 50 ml M9 medium in side-arm flasks which were then incubated at 37° C. and shaken at 180 rpm. The OD₆₁₀ of the growing cultures was monitored regularly and when the OD₆₁₀=0.2 the cultures were placed on ice. Aspartate was added to side-arm flasks to give a final concentration of 3 mM (aspartate stock solution made up in M9 salts).

The test compound or mixtures of compounds were dissolved in ethanol and added to cultures to give the required final concentrations. Negative Controls were prepared with equal volumes of ethanol. Cultures were then placed in a ₃₇° C. incubator and shaken for 4 hours (OD₆₁₀ approximately 0.7) before being removed and put on ice. Samples were then removed for eta-galactosidase assays carried out according to the method of Miller (1972).

V. harveyi Bioassay for the Detection of Al-2 Activity

The V. harveyi bioassay was performed as described previously (Surette and Bassler, 1998). The V. harveyi reporter strain BB170 was grown for 16 hours at 30° C. with shaking in AB medium. Cells were diluted 1:5,000 into 30° C. prewarmed AB medium and 90 ul of the diluted suspension was added to wells containing supernatant. Furanones were added to the wells to achieve the desired final concentrations and the final volume in each well was adjusted with sterile medium to 100 ul. Ten ul of V. harveyi BB152 (Al-1−, Al-2+) supernatant was used as a positive control and 10 ul of E. coli DH5α supernatant or sterile media was used as a negative control. This strain of E. coli has previously been shown to harbor a mutation in the Al-2 synthase gene, ygaG, which results in a truncated protein with no Al-2 activity (Surette et al. 1998). The microliter plates were incubated at 30° C. with shaking at 175 rpm. Hourly determinations of the total luminescence were quantified using the chemiluminescent setting on a Wallac (Gaithersburg, Md.) model 1450 Microbeta Plus liquid scintillation counter. The V. harveyi cell density was monitored by the use of a microplate reader (Bio-Rad, Hercules, Calif.). Activity is reported as the percentage of activity obtained from V. harveyi BB152 cell-free supernatant. While the absolute values of luminescence varied considerably between experiments, the pattern of results obtained was reproducible.

Growth of Staphylococcus aureus

Material and Methods

The growth of Staphylococcus aureus against furanones was tested in sidearm flasks. One percent of an overnight culture was added to the growth media, Nutrient Broth, containing furanones at the concentrations 1-50 μg/ml. The bacteria were incubated at 37 C and growth was measured at 610 nm. The results of these experiments are summarised in the table 1.

TABLE 1
Summary of activity for lactam and other N containing analogues as
inhibitor of AHL-mediated quorum sensing, AI-2 pathway and growth of S.
aureus.

| | | AI-2 | S. aureus |
|---|---|---|---|
| Compound | AHL | (% of control) | (% of control) |
| 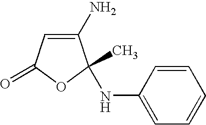 | + + + | 26%, 50 ug/ml 57%, 10 ug/ml 80%, 5 ug/ml | NE at 50 ug/ml |
| 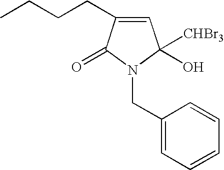 | + + + | 21 %, 50 ug/ml | NE at 50 ug/ml |
| 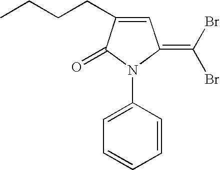 | + + + | | NE at 50 ug/ml |
| 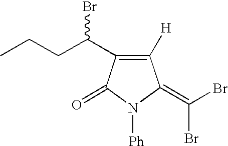 | + + + + | | 0% growth at ug/ml for 10 hrs |
| 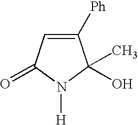 | | 39% (100 ug/ml) | 102% (25 μg/ml) |
| 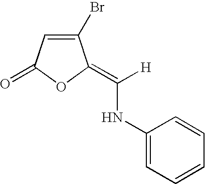 | + + + | 2% (50 μg/ml) | 104% (50 μg/ml) |
| 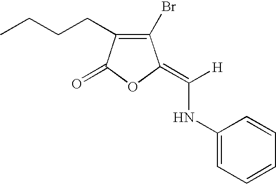 | + + + + | 61% (20 μg/ml) | No effect 50 μg/ml |
| 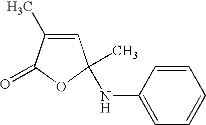 | + + | 50% (100 μg/ml) | No effect 50 μg/ml |
| 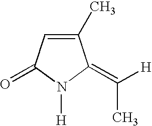 | + + + | | No effect 50 μg/ml |
| 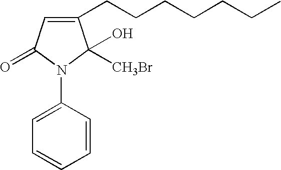 | + + + | | |
| 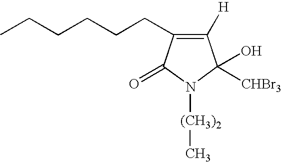 | + + + | | |
| 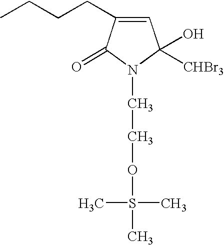 | + + | | No effect (50 μg/ml) |
| 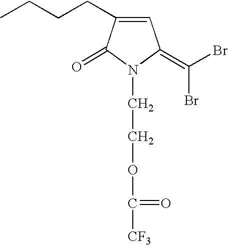 | + + + | | No effect (50 μg/ml) |
| 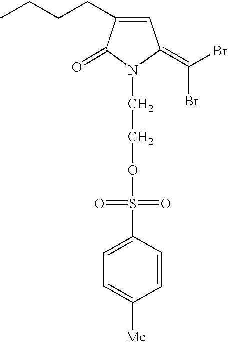 | + + | | No effect (50 μg/ml) |

Other signal regulated phenotypes

| | | | | |
|---|---|---|---|---|
| 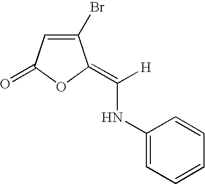 | 40-50% reduction in swimming motility in V. cholerae and V. vulnificus | | 30% reduction in attachment by V. vulnificus | 40% reduction in cholera toxin production by V. cholerae |
| 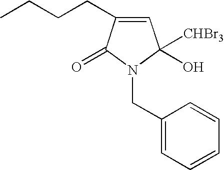 | 40-50% reduction in swimming motility in V. cholerae and V. vulnificus | 50-80% reduction in swimming motility in V. cholerae and V. vulnificus | 30% reduction in attachment by V. vulnificus | 40% reduction in cholera toxin production by V. cholerae |
| 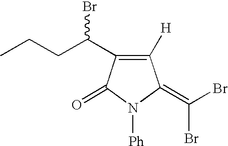 | 40-50% reduction in swimming motility in V. cholerae and V. vulnificus | | 30% reduction in attachment by V. vulnificus | 40% reduction in cholera toxin production by V. cholerae |

Christensen, G. D., W. A. Simpson, J. J. Younger, L. M. Baddour, F. F. Barrett, D. M. Melton, and E. H. Beachey, 1985. Adherence of coagulase-negative staphylococci to plastic tissue culture plates: a quantitative model for the adherence of staphylococci to medical devices. J. Clin. Microbiol. 22(6):99 g 1006.
Andersen, J. B., C. Sternberg, L. K. Poulsen, S. P. Bjom, M. Givskov, and S. Molin. 1998. New unstable variants of green fluorescent protein for studies of transient gene expression in bacteria. Appl. Environ. Microbiol. 64(6):2240-2246.
Jin, T., and M. Inouye. 1993. Ligand binding to the receptor domain regulates the ratio of kinase to phosphatase activities of the signalling domain of the hybrid Escherichia coli transmembrane receptor, Taz1. J. Mol. Biol. 232: 484-49
Miller, J. H. 1972. Experiments in molecular genetics. Cold Spring Harbor Laboratory, Cold Spring Harbor, N.Y.
Surette, M. G., and B. L. Bassler. 1998. Quorum sensing in Escherichia coli and Salmonella typhimurium. Proc. Natl. Acad. Sci., USA 95:7046-7050.
Surette, M. G., M. B. Miller, and B. L. Bassler. 1999. Quorum sensing in Escherichia coli, Salmonella typhimurium, and Vibrio harveyi: a new family of genes responsible for autoinducer production Pro. Natl. Acad. Sci., USA 96:1639-1644.

Any description of prior art documents herein is not to be taken as an admission that the documents form part of the common general knowledge of the relevant art.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Any description of prior art documents herein is not to be taken as an admission that the documents form part of the common general knowledge of the relevant art.

1-117. (canceled)

118. A method for the preparation of compound of formula II 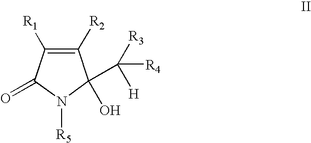
wherein R₁ and R₂ are independently selected from the group H, halogen, substituted or unsubstituted alkyl, substituted or unsubstituted alkoxy, substituted or unsubstituted substituted or unsubstituted oxoalkyl, substituted or unsubstituted substituted or unsubstituted alkenyl, substituted or unsubstituted substituted or unsubstituted aryl or arylalkyl, optionally interrupted by one or more hetero atoms, straight chain or branched chain, hydrophilic or fluorophilic;
R₃ and R₄ are independently selected from the group H, halogen, substituted or unsubstituted alkyl, substituted or unsubstituted alkoxy, substituted or unsubstituted substituted or unsubstituted aryl or substituted or unsubstituted arylalkyl;
R₅ is selected from the group consisting of H, substituted or unsubstituted alkyl, substituted or unsubstituted alkoxy, substituted or unsubstituted alkylsilyl, substituted or unsubstituted substituted or unsubstituted oxoalkyl, substituted or unsubstituted alkenyl, substituted or unsubstituted substituted or unsubstituted aryl or substituted or unsubstituted arylalkyl, optionally interrupted by one or more hetero atoms, straight chain or branched chain, hydrophilic or fluorophilic, or
forms part of an amino acid, or
is a nucleoside, an oligomer, a polymer, a dendrimer, a substrate or a surface,
the method comprising reacting a compound of formula I 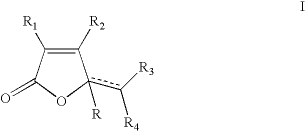
wherein R₁ and R₂ are independently H, halogen, substituted or unsubstituted alkyl, substituted or unsubstituted alkoxy, substituted or unsubstituted oxoalkyl, substituted or unsubstituted alkenyl, substituted or unsubstituted aryl or substituted or unsubstituted arylalkyl, optionally interrupted by one or more hetero atoms, straight chain or branched chain, hydrophilic or fluorophilic;
R₃ and R₄ are independently H, halogen, substituted or unsubstituted alkyl, substituted or unsubstituted alkoxy, substituted or unsubstituted aryl or arylalkyl; and R is hydroxy, halogen; and 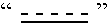
represents a single bond, in which case R is absent, or a double bond, provided that at least one of R₁, R₂, R₃ and R₄ is halogen,
with a compound of formula R₅NH₂
wherein R₅ is selected from the group consisting of H, substituted or unsubstituted alkyl, hydroxy, substituted or unsubstituted alkoxy, substituted or unsubstituted oxoalkyl, substituted or unsubstituted alkenyl, substituted or unsubstituted aryl or substituted or unsubstituted arylalkyl, optionally interrupted by one or more hetero atoms, straight chain or branched chain, hydrophilic or fluorophilic, or
forms part of an amino acid, or
is a nucleoside, an oligomer, a polymer, a dendrimer, a substrate or a surface.

119. A method according to claim 118, wherein, at least one of R₁, R₂, R₃ and R₄ is halogen.

120. A method according to claim 118, wherein R₅ is a residue of a naturally occurring compound.

121. A method according to claim 118, wherein R₅ is a biomolecule.

122. A method according to claim 121, where R₅ is a coenzyme or cofactor.

123. A method according to claim 118, wherein R₅ is an oligomer or a polymer.

124. A method according to claim 123, wherein the oligomer or polymer is a biomolecule.

125. A method according to claim 124, wherein R₅ is a peptide or polyamide.

126. A method according to 118, wherein R₅ is a protein residue.

127. A method according to claim 126, where R₅ is an enzyme or a receptor.

128. A method according to 118, wherein R₅ is an oligomer or polymer comprising nucleic acid residues.

129. A method according to claim 128, wherein R₅ is a polynucleotide.

130. A method according to claim 129, wherein the polynucleotide is DNA or RNA.

131. A method according to claim 118, wherein R₅ is a surface or a substrate with which the nitrogen atom of compound II is associated.

132. A method according to claim 131, wherein the association is chemical bonding.

133. A method according to claim 132, wherein the association is covalent bonding.

134. A method according to 118, wherein the surface or substrate may be biological or synthetic.

135. A compound of formula II: 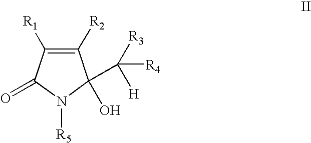
wherein R₁ and R₂ are independently H, halogen, substituted or unsubstituted alkyl, substituted or unsubstituted alkoxy, substituted or unsubstituted oxoalkyl, substituted or unsubstituted alkenyl, substituted or unsubstituted aryl or substituted or unsubstituted arylalkyl, optionally interrupted by one or more hetero atoms, straight chain or branched chain, hydrophilic or fluorophilic;
R₃ and R₄ are independently H, halogen, substituted or unsubstituted alkyl, substituted or unsubstituted alkoxy, substituted or unsubstituted aryl or substituted or unsubstituted arylalkyl;
R₅ is selected from the group consisting of H, hydroxy, substituted or unsubstituted alkyl, substituted or unsubstituted alkoxy, substituted or unsubstituted oxoalkyl, substituted or unsubstituted alkenyl, substituted or unsubstituted aryl, substituted or unsubstituted arylalkyl, optionally interrupted by one or more hetero atoms, straight chain or branched chain, hydrophilic or fluorophilic, or
forms part of an amino acid, or
is a nucleoside, an oligomer, a polymer, a dendrimer, a substrate or a surface.

136. A compound according to claim 135, wherein R₅ is a D- or L-nucleoside.

137. A compound according to claim 135, wherein R₅ is an oligomer or a polymer.

138. A compound according to claim 135, wherein R₅ is dendrimer.

139. A compound according to claim 135, wherein R₅ is a substrate.

140. A compound, according to claim 135, wherein R₅ is a surface.

141. A compound according to claim 135, wherein at least one of R₁, R₂, R₃ and R₄ is halogen.

142. A method for preparing a compound of formula III, the method comprising dehydration a compound of formula II according to claim 135; 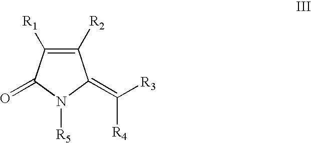
wherein R₁ and R₂ are independently selected from H, halogen, substituted or unsubstituted alkyl, substituted or unsubstituted alkoxy, substituted or unsubstituted oxoalkyl, substituted or unsubstituted alkenyl, substituted or unsubstituted aryl or substituted or unsubstituted arylalkyl, optionally interrupted by one or more hetero atoms, straight chain or branched chain, hydrophilic or fluorophilic;
R₃ and R₄ are independently selected from H, halogen, substituted or unsubstituted alkyl, substituted or unsubstituted alkoxy, substituted or unsubstituted aryl or arylalkyl; and R₅ is selected from the group consisting of H, substituted or unsubstituted alkyl, hydroxy, substituted or unsubstituted alkoxy, substituted or unsubstituted oxoalkyl, substituted or unsubstituted alkenyl, substituted or unsubstituted aryl or substituted or unsubstituted arylalkyl, optionally interrupted by one or more hetero atoms, straight chain or branched chain, hydrophilic or fluorophilic, or
forms part of an amino acid, or
is a nucleoside, an oligomer, a polymer, a dendrimer, a substrate or a surface.

143. A method according to claim 142, wherein at least one of R₁, R₂, R₃ and R₄ in formula III is halogen.

144. A method according to claim 141, wherein the dehydration is carried out in the presence of a dehydrating agent.

145. A method according to claim 144, wherein the dehydrating agent is selected from the group consisting of phosphorus pentoxide, silica gel, molecular sieves, alumina, acidic resins and polymers, phosphorus oxychloride, acetic anhydride, N,N′-dicyclohexylcarbodiimide (DCC), trifluoroacetic acid, sulfuric acid, trifluoroacetic anhydride, and trifluorasulfonic acid anhydride (triflic anhydride).

146. A compound of formula III: 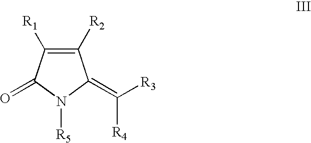
wherein R₁ and R₂ are independently selected from H, halogen, substituted or unsubstituted alkyl, substituted or unsubstituted alkoxy, substituted or unsubstituted oxoalkyl, substituted or unsubstituted alkenyl, substituted or unsubstituted aryl or substituted or unsubstituted arylalkyl, optionally interrupted by one or more hetero atoms, straight chain or branched chain, hydrophilic or fluorophilic;
R₃ and R₄ are independently selected from H, halogen, substituted or unsubstituted alkyl, substituted or unsubstituted alkoxy, substituted or unsubstituted aryl or arylalkyl; and
R₅ is selected from the group consisting of H, substituted or unsubstituted alkyl, hydroxy, substituted or unsubstituted alkoxy, substituted or unsubstituted oxoalkyl, substituted or unsubstituted alkenyl, substituted or unsubstituted aryl or substituted or unsubstituted arylalkyl, optionally interrupted by one or more hetero atoms, straight chain or branched chain, hydrophilic or fluorophilic, or
forms part of an amino acid, or
is a nucleoside, an oligomer, a polymer, a dendrimer, a substrate or a surface.

147. A compound according to claim 144, wherein at least one of R. R₂, R₃ and R₄ is halogen.

148. A compound according to claim 146, selected from the group consisting of:

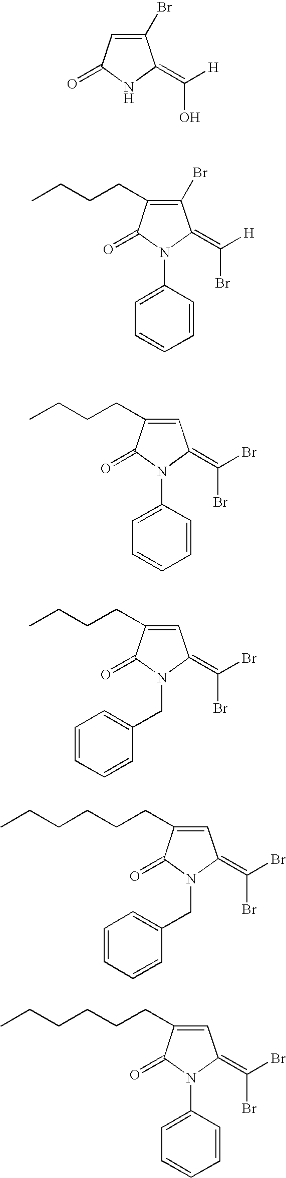

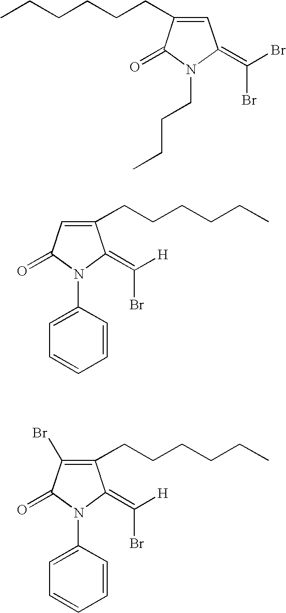

149. A method for the preparation of a compound of formula IV 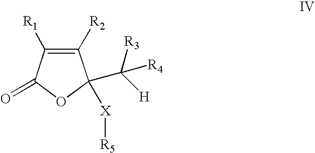
wherein R₁ and R₂ are independently selected from H, halogen, substituted or unsubstituted alkyl, substituted or unsubtituted alkoxy, substituted or unsubstituted oxoalkyl, substituted or unsubstituted alkenyl, substituted or unsubstituted aryl or substituted or unsubstituted arylalkyl, optionally interrupted by one or more hetero atoms, straight chain or branched chain, hydrophilic or fluorophilic;
R₃ and R₄ are independently selected from H, halogen, substituted or unsubstituted alkyl, substituted or unsubstituted alkoxy, substituted or unsubstituted aryl or arylalkyl; and
R₅ is selected from the group consisting of H, substituted or unsubstituted alkyl, hydroxy, substituted or unsubstituted alkoxy, substituted or unsubstituted oxoalkyl, substituted or unsubstituted alkenyl, substituted or unsubstituted aryl or substituted or unsubstituted arylalkyl, optionally interrupted by one or more hetero atoms, straight chain or branched chain, hydrophilic or fluorophilic, or
forms part of an amino acid, or
is a nucleoside, an oligomer, a polymer, a dendrimer, a substrate or a surface,
X is O or NR₆, where R₆ be independently selected from R₁,
the method comprising reacting a compound of formula I as defined in claim 1, wherein R₃ is a hydrogen and 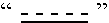
represents a double bond.

150. A method according to claim 149, wherein at least one of R₁, R₂, R₃ and R₄ is halogen.

151. A method according to claim 149, wherein R₅ is H.

152. A compound of formula IV 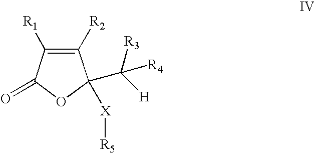
wherein R₁ and R₂ are independently selected from H, halogen, substituted or unsubstituted alkyl, substituted or unsubstituted alkoxy, substituted or unsubstituted oxoalkyl, substituted or unsubstituted alkenyl, substituted or unsubstituted aryl or substituted or unsubstituted arylalkyl,
optionally interrupted by one or more hetero atoms, straight chain or branched chain, hydrophilic or fluorophilic;
R₃ and R₄ are independently selected from H, halogen, substituted or unsubstituted alkyl, substituted or unsubstituted alkoxy, substituted or unsubstituted aryl or arylalkyl;
R₅ is selected from the group consisting of H, substituted or unsubstituted alkyl, hydroxy, substituted or unsubstituted alkoxy, substituted or unsubstituted oxoalkyl, substituted or unsubstituted alkenyl, substituted or unsubstituted aryl or substituted or unsubstituted arylalkyl, optionally interrupted by one or more hetero atoms, straight chain or branched chain, hydrophilic or fluorophilic, or
forms part of an amino acid, or
is a nucleoside, an oligomer, a polymer, a dendnimer, a substrate or a surface; and
X is O or NR₆, where R₆ be independently selected from R₁.

153. A compound according to claim 148, wherein at least one of R₁, R₂, R₃ and R₄ is halogen.

154. A compound according to claim 152, wherein X is NR₆.

155. A compound according to claim 154, wherein R₆ is an optionally substituted arylalkyl.

156. A compound according to claim 152 selected from the group consisting of:

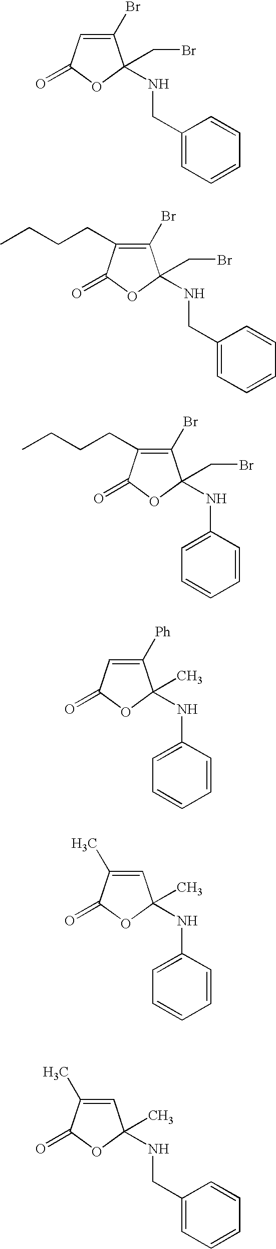

157. A compound of formula V: 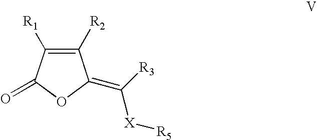
wherein R₁ and R₂ are independently selected from H, halogen, substituted or unsubstituted alkyl, substituted or unsubstituted alkoxy, substituted or unsubstituted oxoalkyl, substituted or unsubstituted alkenyl, substituted or unsubstituted aryl or substituted or unsubstituted arylalkyl, optionally interrupted by one or more hetero atoms, straight chain or branched chain, hydrophilic or fluorophilic;
R₃ is selected from H, halogen, substituted or unsubstituted alkyl, substituted or unsubstituted alkoxy, substituted or unsubstituted aryl or arylalkyl;
X is O or NR₆, where R₆ is as defined above; and
R₅ is selected from the group consisting of H, substituted or unsubstituted alkyl, hydroxy, substituted or unsubstituted alkoxy, substituted or unsubstituted oxoalkyl, substituted or unsubstituted alkenyl, substituted or unsubstituted aryl or substituted or unsubstituted arylalkyl, optionally interrupted by one or more hetero atoms, straight chain or branched chain, hydrophilic or fluorophilic, or
forms part of an amino acid, or
is a nucleoside, an oligomer, a polymer, a dendrimer, a substrate or a surface.

158. A compound of formula (VI): 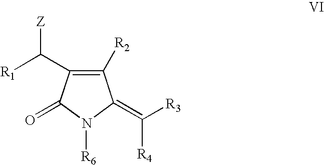
wherein R₁ and R₂ are independently selected from H, halogen, substituted or unsubstituted alkyl, substituted or unsubstituted alkoxy, substituted or unsubstituted oxoalkyl, substituted or unsubstituted alkenyl, substituted or unsubstituted aryl or substituted or unsubstituted arylalkyl, optionally interrupted by one or more hetero atoms, straight chain or branched chain, hydrophilic or fluorophilic;
R₃ and R₄ are independently selected from H, halogen, substituted or unsubstituted alkyl, substituted or unsubstituted aryl or arylalkyl;
R₅ is selected from the group consisting of H, substituted or unsubstituted alkyl, hydroxy, substituted or unsubstituted alkoxy, substituted or unsubstituted oxoalkyl, substituted or unsubstituted alkenyl, substituted or unsubstituted aryl or substituted or unsubstituted arylalkyl, optionally interrupted by one or more hetero atoms, straight chain or branched chain, hydrophilic or fluorophilic, or
forms part of an amino acid, or
is a nucleoside, an oligomer, a polymer, a dendrimer, a substrate or a surface; and
Z is selected from the group R₂, halogen, OC(O)R₂, ═O, amine azide, thiol, R₂, mercaptoaryl, arylalkoxy, mercaptoarylalkyl, SC(O)R₂, OS(O)₂R₂, NHC(O)R₂, ═NR₂ or NHR₂.

159. An oligomer or a polymer formed by oligomerising or polymerising a compound of formula II-VI directly or with one or more other monomers.

160. An oligomer or a polymer according to claim 159, wherein the one or more other monomer is selected from the group acrylate ester such as substituted or unsubstituted alkyl, hydroxyalkyl, aminoalkyl, or substituted substituted or unsubstituted aryl acrylates or methacrylates, crotonates, substituted or unsubstituted acrylonitriles, vinyl alcohols or acetates, styrene and siloxanes.

161. A surface coating or polymer having incorporated therein a compound according to claim 118.

162. Use of a compound according to claim 118 as antimicrobial and/or antifouling agent.

163. Use of a compound according to claim 135 in a medical, scientific and/or biological application(s).

164. A composition comprising at least one compound according to claim 135 and a carrier or diluent.

165. A composition according to claim 164, where the carrier or diluent is a liquid.

166. A composition according to claim 164, where the composition is in the form of a solution or suspension of at least one of the compounds.

167. A composition according to claim 165, wherein the liquid is an aqueous solvent or a non-aqueous solvent.

168. A composition of claim 164, wherein the solvent is a one or more organic solvent(s).

169. A composition according to claim 165, wherein the liquid is an ionic liquid.

170. A composition according to claim 164, in an aerosol or powder formulation.

171. A composition according to 164, including organic or inorganic polymeric substances.

172. A composition according to claim 171, wherein the compound is admixed with a polymer or bound to, or adsorbed on to, a polymer.

173. A composition according to claim 164 formulated as a disinfectant or cleaning formulation.

174. A composition according to claim 164 in the form of a powder, solutions, suspension, dispersion, emulsion or gel.

175. A composition according to claim 164 in the form of a pharmaceutical composition comprising a pharmaceutically acceptable carrier, diluent and/or excipient.

176. A composition according to claim 175, wherein the composition is a form suitable for parenteral or non-parenteral administration.

177. A composition according to claim 176 formulated for topical, intradermal, intramuscular, intraperitoneal, intravenous, subcutaneous, intranasal, epidural, ophthalmic, or oral administration.

178. A composition according to claim 175 formulated for administration by infusion or bolus injection, absorption through epithelial or mucocutaneous linings and may be administered together with other biologically active agents.

179. A composition according to claim 175 formulated for use in an inhaler or nebulizer.

180. A method of treating an infection in a human or animal subject the method comprising administration to the subject of an effective amount a compound in accordance with claim 135.

181. A method according to claim 180 wherein treatment is therapeutic or prophylactic.

182. A method or treating an infection or condition in a subject that is characterised by biofilm formation comprising administering a compound according to claim 135.

183. A method according to claim 182, wherein the condition is cystic fibrosis.

184. A method according to claim 182, wherein the condition is dental caries, periodontitis, otitis media, muscular skeletal infections, necrotising fascitis, biliary tract infection, osteomyelitis, bacterial prostatitis, native valve endocarditis, cystic fibrosis pneumonia, meloidosis.

185. A method according to claim 182, wherein the condition is nosocomial infection.

186. A method according to claim 185, wherein the infection is ICU pneumonia or an infection associated with sutures, exit sites, arteriovenous sites, scleral buckles, contact lenses, urinary catheter cystitis, peritoneal dialysis (CAPD) peritonitis, IUDs, endotracheal tubes, Hickman catheters, central venous catheters, mechanical heart valves, vascular grafts, biliary stent blockage, and orthopedic devices, penile prostheses.

187. A method according to claim 182, wherein the infection is selected from the group a skin infection, burn infection and wound infection.

188. A method according to claim 182, wherein the is an immunocompromised individuals.

189. A method for treating or preventing biofilm formation on a surface, the method comprising contacting the surface with a compound according to claim 135.

190. A method according to claim 189, wherein the surface is a non-biological surface.

191. A method according to claim 189, wherein the surface is a natural surface.

192. A method according to claim 189, wherein the surface is a surface of a plant, seed, wood, fibre or hair.

193. A method according to claim 189, wherein the surface is a biological surface.

194. A method according to claim 193, wherein the surface is a surface of a tissue, membrane or skin.

195. A method according to claim 189, wherein the surface is a hard surface.

196. A method according to claim 195, wherein the surface is formed of a metal, an organic and inorganic polymer, a natural or synthetic elastomer, board, glass, wood, paper, concrete, rock, marble, gypsum and ceramic materials which optionally are coated.

197. A method according to claim 189, wherein the surface is a coating.

198. A method according to claim 197, wherein the coating is an enamel, varnish or paint.

199. A method according to claim 189, wherein the surface is a soft surface.

200. A method according to claim 199, wherein the surface is a surface of a fibre.

201. A method according to claim 200, wherein the fibre is in the form of a yarn, a textile, a vegetable fibre, rock wool.

202. A method according to claim 189, wherein the surface is a porous surface.

203. A method according to claim 189, wherein the surface is a surface of process equipment or components of cooling equipment.

204. A method according to claim 203, wherein the process equipment is for a cooling tower, a water treatment plant, a dairy processing plant, food processing plant, a chemical process plant or a pharmaceutical process plant or a component thereof.

205. A method according to claim 204, wherein the surface is that of a filter.

206. A method according to claim 205, wherein the filter is a membrane filter.

207. A method according to claim 189, wherein the surface is a surface of toilet bowls, bathtubs, drains, highchairs, counter tops, vegetables, meat processing rooms, butcher shops, food preparation areas, air ducts, air-conditioners, carpets, paper or woven product treatment, nappies (diapers), personal hygiene products, and washing machines.

208. A method according to claim 189, wherein the surface is an industrial surface.

209. A method according to claim 189, wherein the surface is a medical surface.

210. A method according to claim 189, wherein the surface is a hospital, veterinary hospital surface, mortuary surface and funeral parlour surface.

211. A dentifrice, a mouthwash or a composition for the treatment of dental caries comprising a compound in accordance with claim 135.

212. A composition far treatment of acne comprising a compound in accordance with claim 135.

213. A composition for cleaning and disinfecting contact lenses comprising a compound in accordance with claim 135.

214. A medical device incorporating a compound as defined below comprising a compound in accordance with claim 135 on at least one surface thereof.

215. An implant device having at least one surface associated with compound as defined below comprising a compound in accordance with claim 135.

216. An implant device according to claim 215, wherein the device is an artificial heart valve or hip joint, an indwelling catheter, pacemaker, surgical pin and the like.

217. An antifouling composition comprising an effective amount of a compound as defined below comprising a compound in accordance with claim 135.

218. An antifouling coating composition, the composition comprising an effective amount of a compound as defined below comprising a compound in accordance with claim 135.

219. An shellfish or aquaculture apparatus having at least one surface associated with a compound as defined below comprising a compound in accordance with claim 135.

220. A biofilm removing or inhibiting composition comprising an amount of a compound according to any one of comprising a compound in accordance with claim 135 and a vehicle or carrier, wherein the amount of the mixture is effective to remove or disrupt a bacterial biofilm or inhibit normal biofilm formation.

221. A composition according to claim 220, additionally comprising a surfactant selected from group consisting of anionic, nonionic, amphoteric, biological surfactants and mixtures thereof.

222. A composition of claim 221 further comprising a compound selected from the group consisting of biocides, fungicides, antibiotics, and mixtures thereof.

223. A method of removing biofilm from a surface comprising the step of administering a cleaning-effective amount of a compound of claim comprising a compound in accordance with claim 135 to a biofilm-containing surface.

224. A method of preventing biofilm formation on a surface comprising the step of administering an effective amount of the compound as defined below comprising a compound in accordance with claim 135 to a surface, wherein the amount is effective to prevent biofilm formation.

225. A method of claim 224, wherein the surface is a hard, rigid surface.

226. A method of claim 224, wherein the surface is selected from the group consisting of a drainpipe, glaze ceramic, porcelain, glass, metal, wood, chrome, plastic, vinyl, and formica.

227. A method of claim 224, wherein the surface is a soft, flexible surface.

228. A method of claim 224, wherein the surface is selected from the group consisting of shower curtains or liners, upholstery, laundry, and carpeting.

229. A method of claim 224, wherein the biofilm is produced by a bacteria of the class Pseudomonas.

230. The method of claim 224, wherein the bacteria is of the species Pseudomonas aeuroginosa.

231. A method of claim 224, wherein the biofilm is produced by an organism selected from the group consisting of bacteria, algae, fungi and protozoa.

232. A dentifrice comprising an effective amount of a compound as defined below comprising a compound in accordance with claim 135, wherein the amount is effective to either prevent or remove biofilm formation.

233. A mouthwash comprising an effective amount of a compound as defined below comprising a compound in accordance with claim 135, wherein the amount is effective to either prevent or remove biofilm formation.

234. An optical lens, wherein at least a part of a surface of the lens is associated with a compound as defined below comprising a compound in accordance with claim 135.

235. An optical lens according to claim 234, wherein the lens in a contact lens.
